(19) United States
(12) Patent Application Publication
(10) Pub. No.: US 2007/0015742 A1
(43) Pub. Date: Jan. 18, 2007

(54) Porphyrin derivatives

(76) Inventor: Gokhan Yahioglu, Bristol (GB)

Correspondence Address: FISH & RICHARDSON PC, P.O. BOX 1022, MINNEAPOLIS, MN, 55440-1022, US

(21) Appl. No.: 10/547,061

(22) PCT Filed: Feb. 26, 2004
(86) PCT No.: PCT/GB04/00773

§ 371 Date: Aug. 25, 2005

(30) Foreign Application Priority Data
Feb. 26, 2003 (GB) 0304456.7

(51) Int. Cl. C07D 487/22; A61K 31/555; A61K 31/4439; A61K 31/409

(52) U.S. Cl. 514/185; 514/338; 514/410; 540/145; 540/466

(57) ABSTRACT

The present invention relates to a compound of formula (I): Formula (I) wherein one or two of A, B, C and D are each independently selected from S, O, Se and Te, and the remainder are N; a, b, c and d are each independently substituted or unsubstituted 5-membered heterocyclic groups having the members necessary to complete a porphyrin, chlorin, bacteriochlorin or isobacteriochlorin nucleus in which one or two of the nitrogens are replaced by S, O, Se or Te; M is H or a metal; R₁, R₂, R₃ and R₄ are each independently selected from: H; alkyl; cycloalkyl; halogen; aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, COOH, COO-alkyl, —OZ, —COOZ, a polyethylene glycol group, an alkyl sulfonate group, an alkyl-COOH group, a substituted or unsubstituted benzyl group, a sugar derivative, —C≡C—(CH₂)ₚCO₂R₁₀, where R₁₀ is H or alkyl, and O(CH₂)ᵣCOR₁₁, where R₁₁, is OH, O-alkyl or —N-succinimide, and p and r are each independently an integer from 1 to 10; Formula (II) wherein W is an aryl, alkyl or heteroaryl group, each of which may be optionally substituted by one or more substituents listed above where Z is a silicon-containing protecting groups; and wherein when a, b, c and d have the members necessary to complete a porphyrin nucleus in which one or two of the nitrogens are replaced by S, O, Se or Te, (c) R₁, R₂ and R₃ are identical, and R₄≠R₁, R₂, R₃; or (d) R₁=R₃; R₂=R₄, where R₁, R₃≠R₂, R₄; or (d) R₂=R₃; R₁≠R₄; and R₁, R₄≠R₂, R₃.

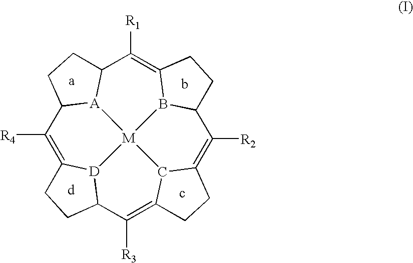

The present invention relates to core-modified porphyrin derivatives and pharmaceutical compositions thereof. More specifically, but not exclusively, the invention relates to core-modified porphyrin derivatives having applications in the field of photodynamic therapy.

### BACKGROUND

Porphyrins have found uses in numerous applications including precursors for novel conducting polymers [Wagner et al, J. Am. Chem. Soc., 1994, 116, 9759; Anderson, Inorg. Chem., 1994, 33, 972 and Arnold et al, Tetrahedron, 1992, 48, 8781]; non-linear optically active (NLO) materials [Anderson et al, Angew. Chem. Int. Ed. Engl., 1994, 33, 655 and Arnold et al, J. Am. Chem. Soc., 1993, 115, 12197]; photosynthetic model compounds [Wagner et al, J. Org. Chem., 1995, 60, 5266, and Lin et al, Science, 1994, 264, 1105]; and enzyme mimics [Anderson et al, Angew. Chem. Int. Ed. Engl., 1990, 29, 1400; Anderson et al, J. Chem. Soc., Chem. Commun., 1992, 946 and Mackay et al, J. Chem. Soc., Chem. Commun., 1992, 43]. Meso-tetraalkynyl-substituted porphyrins are reported by Anderson in Tetrahedron Lett., 1992, 33 1101. Porphyrins have also been the focus of investigations in the field of photodynamic therapy.

Replacing one or more of the four pyrrolic nitrogen atoms in a porphyrin with heteroatoms such as oxygen, sulphur, selenium and tellurium leads to new “core-modified” porphyrin derivatives. Such core-modified derivatives have altered metal coordination properties [Latos-Grazynski, L. et al, New J. Chem. 1997, 21, 691], acid-base strength [Broadhurst, M. J. et al, J. Chem. Soc. C 1971, 3681], redox potentials [Pandian, R. P. et al, Inorg. Chem. 1994, 33, 3317], electronic energy levels [Gopinath, C. S. et al, J. Chem. Soc. Dalton Trans. 1996, 1255] and excited state lifetimes [Ulman, A. et al, Tet. Lett., 1978, 1885; Ha, J-H et al, Chem. Phys. Lett., 2001, 349, 271].

The effects of heteroatom substitution and meso aryl group substitution on the physical and photophysical properties and on the biological properties of core modified porphyrins has been investigated by a number of groups [Stilts, C. E. et al, J. Med. Chem., 2000, 43, 2403; Hilmey, D. G. et al, J. Med. Chem., 2002, 45, 449]. Other groups [arcinkowska, E. et al, Anticancer Res. 1997, 17, 3313; Ziolkowski, P. et al, J. Cancer. Res. Clin. Oncol., 1999, 125, 563] have reported 21-thia-21-23-dithiatetraphenylporphyrins and 21-oxatetraphenylporphyrins as photosensitisers.

The synthesis of symmetrical and unsymmetrical meso-substituted heteroatom-substituted porphyrins was originally developed by Ulman and Manassen [Ulman, A. et al, J. Am. Chem. Soc., 1975, 97, 6540; J. Chem.Soc. Perkin Trans 1, 1979, 1066]. Tetra-phenyl N₃S porphryins were subsequently reported by Latos-Grazynski and Chmielewski [Chimielewski, P. et al, J. Inorg. Chem., 1989, 28, 3456]. The latter approach to the synthesis of monothiaporphyrins allowed for the introduction of unsymmetrical substituents on the porphyrin periphery. The synthesis of monothiaporphyrins was later improved by Srinivasan [Srinivasan, A. et al, Tet. Lett., 1997, 38, 4149], and Cho [Cho, W-S. et al, J Org. Chem., 1999, 64, 7890]. However, all of the aforementioned synthetic approaches result in the synthesis of meso-tetraphenyl substituted core-modified porphyrins.

The present invention seeks to provide new core-modified porphyrins and derivatives thereof, particularly those which exhibit improved properties with regard to photodynamic therapy and/or medical imaging. The invention also seeks to provide a more flexible and improved synthetic strategy for the synthesis of core-modified porphyrins and derivatives thereof.

### STATEMENT OF INVENTION

A first aspect of the invention relates to a compound of formula I, or a pharmaceutically acceptable salt thereof,

A compound of formula I, or a pharmaceutically acceptable salt thereof,

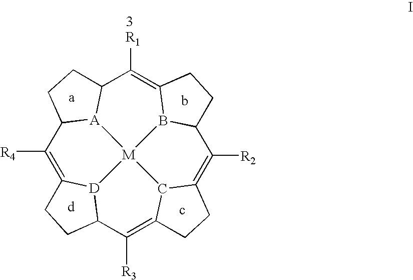

wherein

one or two of A, B, C and D are each independently selected from S, O, Se and Te, and the remainder are N;

a, b, c and d are each independently substituted or unsubstituted 5-membered heterocyclic groups having the members necessary to complete a porphyrin, chlorin, bacteriochlorin or isobacteriochlorin nucleus in which one or two of the nitrogens are replaced by S, O, Se or Te;

M is H or a metal;

R₁, R₂, R₃ and R₄ are each independently selected from: H;
alkyl;
cycloalkyl;
halogen;
aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, COOH, COO-alkyl, —OZ, —COOZ, a polyethylene glycol group, an alkyl sulfonate group, an alkyl-COOH group, a substituted or unsubstituted benzyl group, a sugar derivative, —C≡C—(CH₂)ₚCO₂R₁₀, where R₁₀ is H or alkyl, and O(CH₂)ᵣCOR₁₁, where R₁₁, is OH, O-alkyl or N-succinimide, and p and r are each independently an integer from 1 to 10; 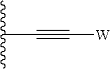
wherein W is an aryl, alkyl or heteroaryl group, each of which may be optionally substituted by one or more substituents selected from OH, CN, CF₃, alkyl, alkoxy, halogen, an isothiocyanate group, a haloacetamide, maleimide, NH₂, NO₂, CONH₂, haloalkyl, COOH, COO-alkyl, —OZ′, —COOZ′, a polyethylene glycol group, an alkyl sulfonate group, an alkyl-COOH group, a substituted or unsubstituted benzyl group, a sugar derivative, —C≡C— (CH₂)ₚCO₂R₁₂, where R₁₂ is H or alkyl, and O(CH₂)ᵣCOR₁₃, where R₁₃ is OH, O-alkyl or N-succinimide, and p′ and r′ are each independently an integer from 1 to 10;
where Z and Z′are each independently silicon-containing protecting groups; and wherein when a, b, c and d have the members necessary to complete a porphyrin nucleus in which one or two of the nitrogens are replaced by S, O, Se or Te,
(a) R₁, R₂ and R₃ are identical, and R₄≠R₁, R₂, R₃; or
(b) R₁=R₃; R₂=R₄, where R₁, R₃ ≠R₂, R₄; or
(c) R₂ =R₃; R₁≠R₄; and R₁, R₄≠R₂, R₃.

A second aspect of the invention relates to a pharmaceutical composition comprising a compound of formula I admixed with a pharmaceutically acceptable diluent, excipient or carrier.

A third aspect of the invention relates to a conjugate molecule comprising a compound of formula I and a targeting moiety selected from a recombinant antibody, a Fab fragment, a F(ab′)₂ fragment, a single chain Fv, a diabody, a disulfide linked Fv, a single antibody domain and a CDR.

A fourth aspect of the invention relates to a conjugate molecule which comprises a polypeptide carrier comprising at least one alpha helix having synthetically attached thereto a plurality of compounds of formula I.

A fifth aspect relates to the use of a compound of formula I, or a conjugate of the invention, in medicine.

A sixth aspect relates to the use of a compound of formula I, or a conjugate of the invention, for medical imaging.

A seventh aspect relates to the use of a compound of formula I, or a conjugate of the invention, in the preparation of a medicament for photodynamic therapy.

An eighth aspect relates to the use of a compound of formula I, or a conjugate of the invention, in the preparation of a medicament for treating a proliferative disorder.

A ninth aspect relates to the use of a compound of formula I in the preparation of a conjugate of the invention.

A tenth aspect relates to a method of treating a proliferative disorder, said method comprising administering to a subject a therapeutic amount of a compound of formula I, or a conjugate of the invention.

An eleventh aspect of the invention relates to a process for preparing a compound of formula I.

### DETAILED DESCRIPTION

As mentioned above, a first aspect of the invention provides compounds of formula I.

As used herein, the term “hydrocarbyl” refers to a group comprising at least C and H that may optionally comprise one or more other suitable substituents. Examples of such substituents may include halo-, alkoxy-, nitro-, an alkyl group, or a cyclic group. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon.

As used herein, the term “alkyl” refers to a saturated carbon-containing chain which may be straight or branched, and substituted (mono- or poly-) or unsubstituted. Preferably, the alkyl group is a branched or unbranched C₁₋₃₀ alkyl group, more preferably an unbranched C₁₋₂₀ alkyl group, even more preferably a C₁₋₁₀ or C₁₋₅ alkyl group. Suitable substituents may include, for example, halo, NO₂, NH₂, alkoxy, OH and COOH.

Accordingly, the term “haloalkyl” refers to an alkyl group as defined above substituted by a halogen, for example, chlorine, bromine, fluorine or iodine.

As used herein, the term “aryl” refers to a substituted (mono- or poly-) or unsubstituted monoaromatic or polyaromatic system, wherein said polyaromatic system may be fused or unfused.

As used herein, the term “heteroaryl” refers to an aromatic heterocycle comprising one or more heteroatoms and which may be substituted (mono- or poly-) or unsubstituted. Said heteroaryl group may be a monoaromatic or polyaromatic system, wherein said polyaromatic system may be fused or unfused. Preferred heteroaryl groups include pyrrole, pyrimidine, pyrazine, pyridine, quinoline and furan.

As used herein, the term “sugar derivative” refers to a derivative of a mono-, di- or tri-saccharide. Monosaccharides can exist as either straight chain or ring-shaped molecules and are classified according to the number of carbon atoms they possess; trioses have three carbons, tetroses four, pentoses five and hexoses six. Each of these subgroups may be further divided into aldoses and ketoses, depending on whether the molecule contains an aldehyde group (—CHO) or a ketone group (C═O). Typical examples of monosaccharides include glucose, fructose, and galactose. Disaccharides consist of two linked monosaccharide molecules, and include for example, maltose and lactose. Trisaccharides consist of three linked monosaccharide molecules.

As used herein, the term “polyethylene glycol group” (PEG) refers to a polyether chain. n the case where R₁, R₂, R₃ or R₄ are substituted by a polyethylene glycol group (PEG), or W is substituted by a polyethylene glycol group, the polyether typically has a molecular weight of 2000 to 5000 Daltons. The polyether may be etherified or esterified at the terminal hydroxy group, are is more preferably etherified or esterified with a methyl group. In one particularly preferred embodiment of the invention, the polyethylene glycol group is of the formula —(OCH₂CH₂)ₜ-T, where T is a terminator group such as an ether or an ester functionality and t is an integer from 1 to 50, preferably 1 to 20, more preferably 1 to 10. In one especially preferred embodiment of the invention, the polyethylene glycol group is of the formula —(OCH₂CH₂)₆—OMe.

In one preferred embodiment, for said compounds of formula I, a, b, c and d are each independently substituted or unsubstituted 5-membered heterocyclic groups having the members necessary to complete a chlorin, bacteriochlorin or isobacteriochlorin nucleus in which one or two of the nitrogens are replaced by S, O, Se or Te

Another preferred embodiment of the invention relates to compounds of formula I wherein:

one or two of A, B, C and D are each independently selected from S, O, Se and Te, and the remainder are N;

a, b, c and d are each independently substituted or unsubstituted 5-membered heterocyclic groups having the members necessary to complete a porphyrin, chlorin, bacteriochlorin or isobacteriochlorin nucleus in which one or two of the nitrogens are replaced by S, O, Se or Te;

M is H or a metal;

R₁, R₂, R₃ and R₄ are each independently selected from:
H;
alkyl;
cycloalkyl;
halogen;
aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, —OZ, —COOZ, a polyethylene glycol group, an alkyl sulfonate group, an alkyl-COOH group, a substituted or unsubstituted benzyl group, and a sugar derivative; 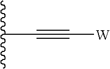
wherein W is an aryl, alkyl or heteroaryl group, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ′, COOZ′, a polyethylene glycol group, an alkyl sulfonate group, an alkyl-COOH group, a substituted or unsubstituted benzyl group, and a sugar derivative;

where Z and Z′ are each independently silicon-containing protecting groups; and wherein when a, b, c and d have the members necessary to complete a porphyrin nucleus in which one or two of the nitrogens are replaced by S, O, Se or Te,
(a) R₁, R₂ and R₃ are identical, and R₄≠R₁, R₂, R₃; or
(b) R₁=R₃; R₂=R₄, where R₁, R₃≠R₂, R₄; or
(b) R₂=R₃; R₁≠R₄; and R₁, R₄R₂, R₃.

Preferably, one of A, B, C and D is S and the remainder are all N.

In another preferred embodiment, R₁, R₂, R₃ and R₄ are each independently selected from:
H;
halogen;
phenyl or pyridyl, each of which may be optionally substituted by one or more substituents selected from OH, CN, CF₃, alkyl, alkoxy, haloalkyl, halogen, NH₂, NO₂, CONH₂, haloalkyl, COOH, COO-alkyl, OZ, COOZ, a polyethylene glycol group, O(CH₂)ᵣCOR₁₁ and —C≡C—(CH₂)ₚCO₂R₁₀; 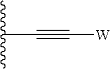
wherein W is a phenyl or pyridyl group, each of which may be optionally substituted by one or more substituents selected from OH, CN, CF₃, alkyl, alkoxy, haloalkyl, halogen, NH₂, NO₂, CONH₂, haloalkyl, COOH, COO-alkyl OZ′, COOZ′, a polyethylene glycol group, —C≡C—(CH₂)_{p′}CO₂R₁₂ and O(CH₂)_{r′}COR₁₃.

In another preferred embodiment, R₁, R₂, R₃ and R₄ are each independently selected from:
H;
halogen;
phenyl or pyridyl, each of which may be optionally substituted by one or more substituents selected from OH, CN, CF₃, alkyl, alkoxy, haloalkyl, halogen, NH₂, NO₂, CONH₂, haloalkyl, COOH, COO-alkyl, OZ, COOZ, —C≡C—(CH₂)ₚCO₂R₁₀ and O(CH₂)ᵣCOR₁₁;

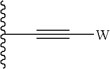

wherein W is a phenyl or pyridyl group, each of which may be optionally substituted by one or more substituents selected from OH, CN, CF₃, alkyl, alkoxy, haloalkyl, halogen, NH₂, NO₂, CONH₂, haloalkyl, COOH, COO-alkyl OZ′, COOZ′, a polyethylene glycol group and O(CH₂)_{r′}COR₁₃.

More preferably, W is an optionally substituted phenyl group.

In an alternative preferred embodiment, W is an optionally substituted pyridyl group.

In one preferred embodiment, W is selected from the following:

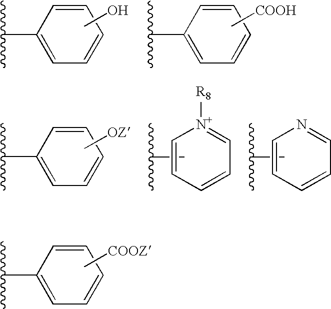

wherein R₈ is an alkyl group, an alkyl sulfonate group, an alkyl-COOH group or a substituted or unsubstituted benzyl group.

Compounds in which R₈ is an alkyl-COOH group may be obtained by reacting the pyridinyl compound with the corresponding bromo- or iodo-alkyl acid.

Compounds in which & is a substituted or unsubstituted benzyl group may be obtained by reacting the pyridinyl compound with a benzyl bromide or a substituted benzyl bromide. Suitable silicon-containing protecting groups will be familiar to the skilled artisan (see for example, “Protective Groups in Organic Synthesis” by Peter G. M. Wuts and Theodoro W. Greene, 2 Edition).

In one preferred embodiment, each silicon-containing protecting group, Z and Z′, is independently (CH₂)_{q}Si(R₅)(R₆)(R₇), wherein R₅, R₆ and R₇ are each independently hydrocarbyl groups and q is 0, 1, 2, 3, 4 or 5.

More preferably, R₅, & and R₇ are each independently alkyl groups.

In one preferred embodiment, q is zero and each of Z and Z′ is independently a trimethylsilyl group (SiMe₂), a triethylsilyl group (SiEt₃), a teritiary-butyldimethylsilyl (TBDMS) group (Si(Me)₂CMe₃), an iso-propyldimethylsilyl group (Si(Me)₂CHMe₂), a phenyldimethylsilyl group (Si(e)₂Ph), a di-tertiary-butylnethylsilyl (DTBMS) group (^{t}Bu₂MeSi) or a tri-isopropylsilyl (TIPS) group (SiⁱPr₃).

In another preferred embodiment, q is other than zero, and each of Z and Z′ is independently a 2-(trimethylsilyl)-ethoxymethyl (SEM) group (CH₂OCH₂CH₂SiMe₃), a tri-iso-propylsilylmethyl group (CH₂SiⁱPr₃), or a 2-(trirnethylsilyl)ethyl (TMSE) group (CH₂CH₂SiMe₃).

In one particularly preferred embodiment, each of Z′ and Z is independently CH₂CH₂SiMe₃.

In a particularly preferred embodiment, W is an aryl group bearing a COOZ′ substituent, wherein Z′ is CH₂CH₂SiMe₃.

In another particularly preferred embodiment, W is selected from the following:

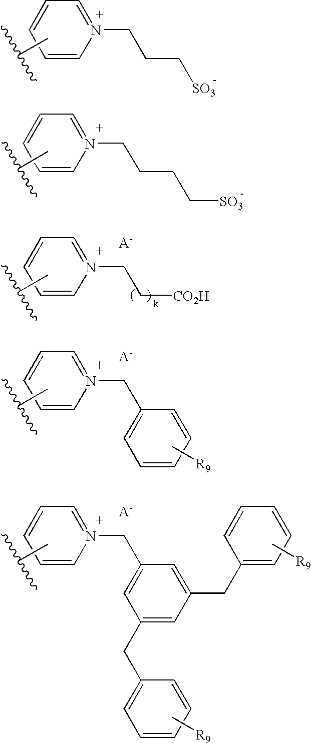

where A⁻ is a counter ion, for example, a halide counter ion such as iodide, or more preferably chloride, k is an integer from 1 to 10, and R₉ is a substituent selected from alkyl, halogen, NO₂, CN, OH, OMe, NH₂, CF₃, COOH and CONH₂.

Compounds in which R₉ is an alkyl-COOH group may be obtained by reacting the pyridinyl compound with the corresponding bromo- or iodo-alkyl acid. Compounds in which Rg is a substituted or unsubstituted benzyl group may be obtained by reacting the pyridinyl compound with the corresponding benzyl bromide. In one especially preferred embodiment, W is selected from:

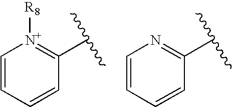

In one preferred embodiment, R₁, R₂, R₃ and R₄ are each independently selected from:
H;
halogen;
phenyl or pyridyl, each of which may be optionally substituted by one or more substituents selected from alkoxy and halogen; 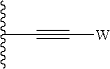
wherein W is unsubstituted phenyl or unsubstituted pyridyl.

In one preferred embodiment of the invention, one or two of A, B, C and D are S, and the remainder are N.

Even more preferably, one of A, B, C and D is S, and the remainder are N.

Preferably, where one or more of R₁, R₂, R₃ and R₄ is an aryl group and said group is substituted by one or more —C≡C—(CH₂)ₚCO₂R₁₀, groups, then p is an integer from 1 to 5. More preferably, p is 3. Preferably, R₁₀ is H. More preferably still, p is 3 and R₁₀ is H, i.e. the substituent is —C≡C—CH₂CH₂CH₂CO₂H.

Preferably, where one or more of R₁, R₂, R₃ and R₄ is an aryl group and said group is substituted by one or more O(CH₂)ᵣCOR₁₁ groups, then r is is an integer from 1 to 5. More preferably, r is 1. Preferably, R₁₁ is OH, OEt or —N-succinimide. More preferably still, r is 1 and R₁₁ is OH, OEt or —N-succinimide, i.e. the substituent is OCH₂COOH, OCH₂COOEt or OCH₂CO—N-succinimide.

Preferably, where one or more of R₁, R₂, R₃ and R₄ is

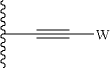

and said W group is an aryl or heteroaryl group substituted by one or more —C≡C—(CH₂)_{p′}CO₂R₁₂, groups, then p′ is an integer from 1 to 5. More preferably, p′ is 3, Preferably, R₁₂ is H. More preferably still, p′ is 3 and R₁₂ is H. i.e. the substituent is —C≡C—CH₂CH₂CH₂CO₂H.

Preferably, where one or more of R₁, R₂, R₃ and R₄ is

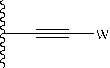

and said W group is an aryl or heteroaryl group substituted by one or more O(CH₂)_{r′}COR₁₃ groups, then r′ is an integer from 1 to 5. More preferably, r′ is 1. Preferably, R₁₃ is OH, Ot or —N-succinimide. More preferably still, r′ is 1 and R₁₃ is OH, OEt or —N-succinimide, i.e. the substituent is OCH₂COOH, OCH₂COOEt or OCH₂CO—N-succinimide.

Core-Modified Chlorins, Bacteriochlorins and ISO-Bacteriochlorins

One embodiment of the invention provides compounds in which the core-modified porphyrin is reduced to a core-modified chlorin, bacteriochlorin or isobacteriochlorin. Such reduction leads to changes in optical properties which result in more efficient absorption in the red and near-infrared regions of the spectrum which render the compounds particularly suitable for PDT.

As used herein, the terms “chlorin”, “bacteriochlorin” and “isobacteriochlorin” refer to macrocycles having the following basic core structures:

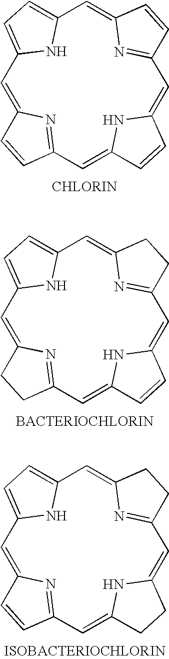

Accordingly, the term “core-modified” chlorin, bacteriochlorin and isobacteriochlorin is used herein to refer to chlorins, bacteriochlorins and isobacteriochlorins in which at least one of the pyrrolic nitrogens is replaced by another heteroatom, for example S, Se, Te or O.

Conventional chlorins and bacteriochlorins are well documented in the art. However, there are a number of disadvantages associated with these species. Firstly, they are synthetically challenging and their structural complexity often leads to a mixture of products. Secondly, they tend to exhibit poor water solubility which means that complex pharmaceutical formulations are required for systemic applications. Thirdly, their chemical instability reduces shelf life, and their negative or neutral overall charge often makes absorption by cells difficult.

To date, there have been no reports in the literature of core-modified chlorin, bacteriochlorin and isobacteriochlorin derivatives as presently claimed. Nor has there been any disclosure of the therapeutic potential of such species as PDT agents.

One embodiment of the invention therefore relates to compounds of formula Ia, i.e., compounds in which at least one of a, b, c and d is in the form of

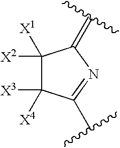

wherein X₁-X₄ are each independently selected from H, OH, alkyl, alkoxy; or C═O, where X₂ and X4 respectively are absent.

In a preferred embodiment, one of a, b, c and d is in the form of

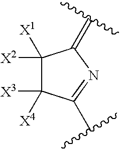

In a preferred embodiment of the invention,

(a) R₁, R₂ and R₃ are identical, and R₄≠R₁, R₂, R₃; or

(b) R₁=R₃; R₂=R₄, where R₁, R₃≠R₂, R₄; or

(c) R₂=R₃; R₁≠R₄; and R₁, R₄≠R₂,R₃.

One particularly preferred embodiment of the invention relates to a compound of formula III or IV

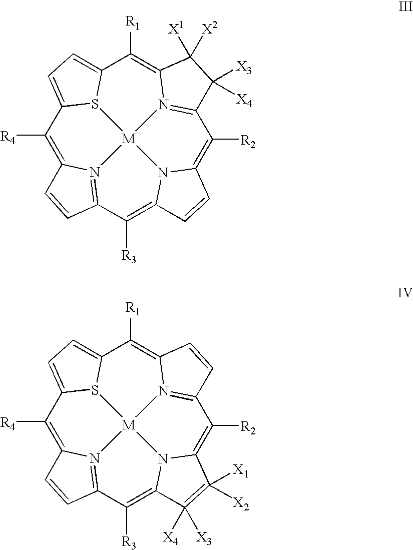

wherein X₁-X4 are each independently selected from H, OH, alkyl, alkoxy; or C═O, where X₂ and X₄ respectively are absent, and R₁-R₄ and M are as defined above.

In a more preferred embodiment, X₁ and X₃ are OH, and X₂ and X₄ are H.

Another preferred embodiment of the invention relates to compounds in which two of a, b, c and d are in the form of

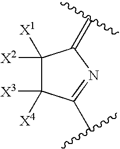

Another particularly preferred embodiment relates to a compound of formula V or VI

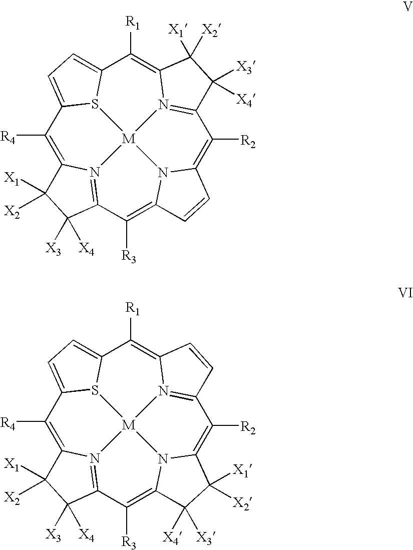

wherein R₁-R₄ and M are as defined above, and X₁l-X₄ and X_{1′}-X_{4′} are each independently selected from H, OH, alkyl, alkoxy, or C═O, where X₂, X₄, X_{2′} and X_{4′} respectively are absent.

More preferably, X₁I, X₃, X₁′ and X₃′ are OH, and X₂, X₄, X₂′ and X₄′ are all H.

In one preferred embodiment, where the compound is of formula III, IV, V or VI, R₁, R₂ and R₃ are the same and are all H, halogen or

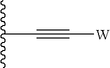

R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl or or (CO)ₙ(O)ₘZ.

Even more preferably, where the compound is of formula III, IV, V or VI, R₁, R₂ and R₃ are all H, halogen or

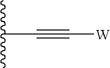

where W is pyridyl; and

R₄ is a halogen substituted aryl group.

More preferably still, the compound is selected from:

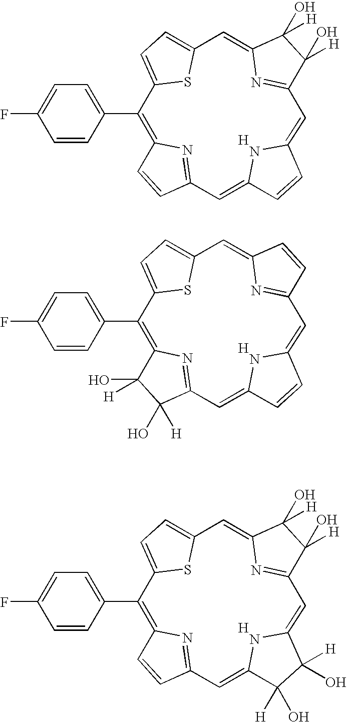

In another preferred embodiment, where said compound is of formula III, IV, V or VI, R₂ and R₃ are the same and are both H, halogen or

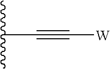

R₁ and R₄ are different and are aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOZ.

Even more preferably, for this embodiment,

R₂ and R₃ are both H, halogen or

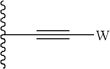

where W is pyridyl;

R₄ is phenyl; and

R₁ is alkoxy substituted phenyl.

More preferably still, the compound is selected from:

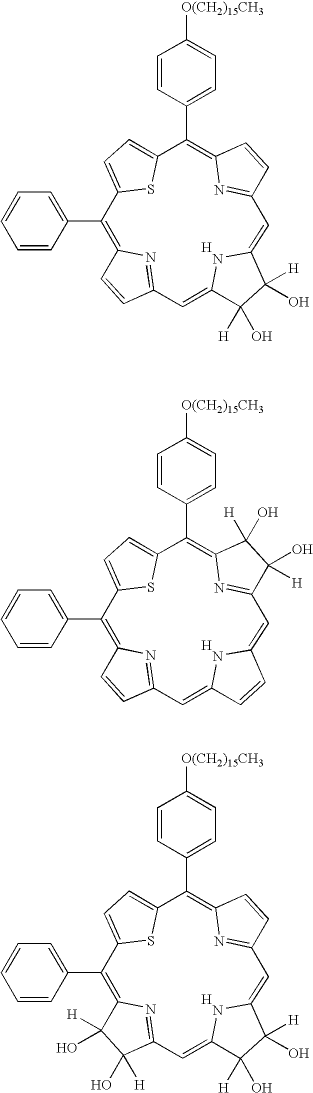

In yet another preferred embodiment, where said compound is of formula ml, IV, V or VI, R₁ and R₃ are the same and are both aryl or heteroaryl, each of which may be optionally substituted by one or more substituents selected from OH, CN, CF₃, alkyl, aLkoxy, haloalkyl, halogen, an isothiocyanate group, a haloacetamide, maleimide, NH₂, NO₂, CONH₂, haloalkyl, COOH, COO-alkyl, OZ and COOZ and

R₂ and R₄ are the same and are both H, halogen or

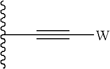

Preferably, for this embodiment,

R₁ and R₃ are both phenyl; and

R₂ and R₄ are both H.

Even more preferably, said compound is selected from the following: 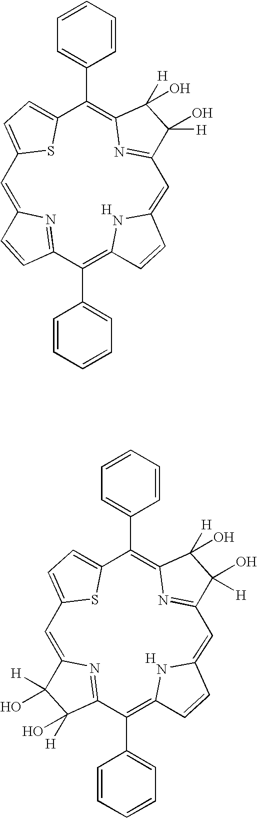
Core Modified Porphyrin Derivatives

In one preferred embodiment, the compound of the invention is a core-modified porphyrin of formula II

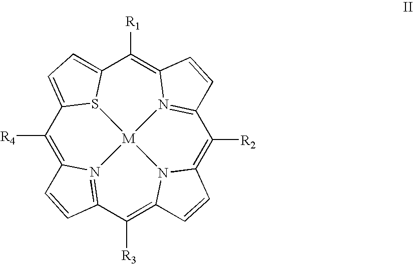

In one particularly preferred embodiment,

R₁ and R₄ are different and are selected from aryl and heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl OZ and COOZ; and

R₂ and R₃ are the same and are both H, halogen or

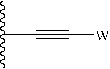

Even more preferably, for this embodiment,

R₁ is aryl optionally substituted by an alkoxy group;

R₂ and R₃ are both H, halogen or

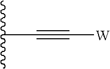

where W is a pyridyl;

R₄ is phenyl.

More preferably still, for this embodiment, the compound is selected from the following:

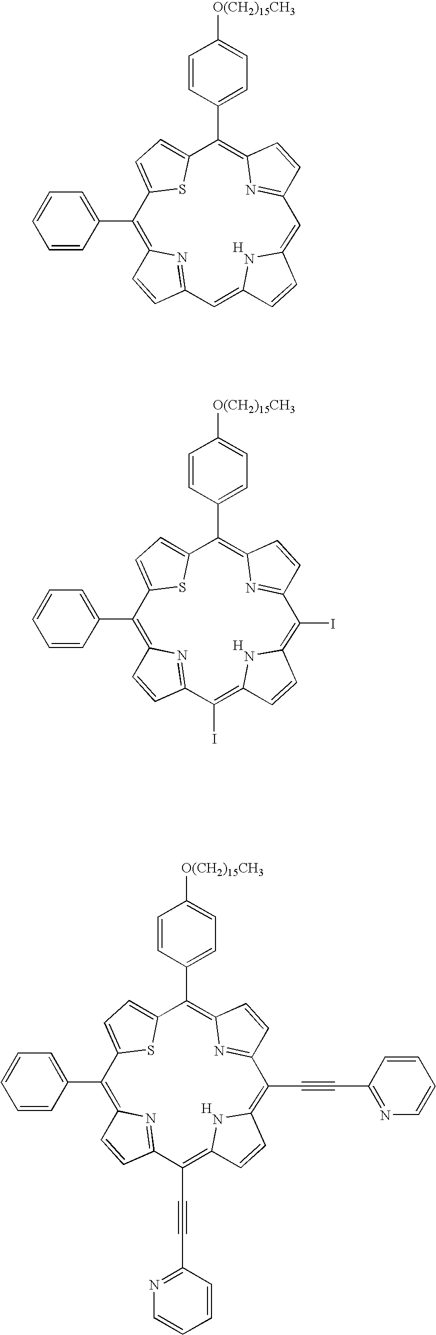

Another preferred embodiment of the invention relates to a compound of formula II wherein R₁ are R₃ are the same and are both H, halogen or

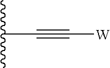

R₂ and R₄ are the same and are both aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl OZ and COOZ.

Preferably, in respect of this embodiment,

R₁ and R₂ are both H, halogen or

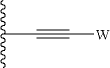

where W is pyridyl;

R₂ and R₄ are both phenyl.

Even more preferably, the compound of formula II is selected from the following:

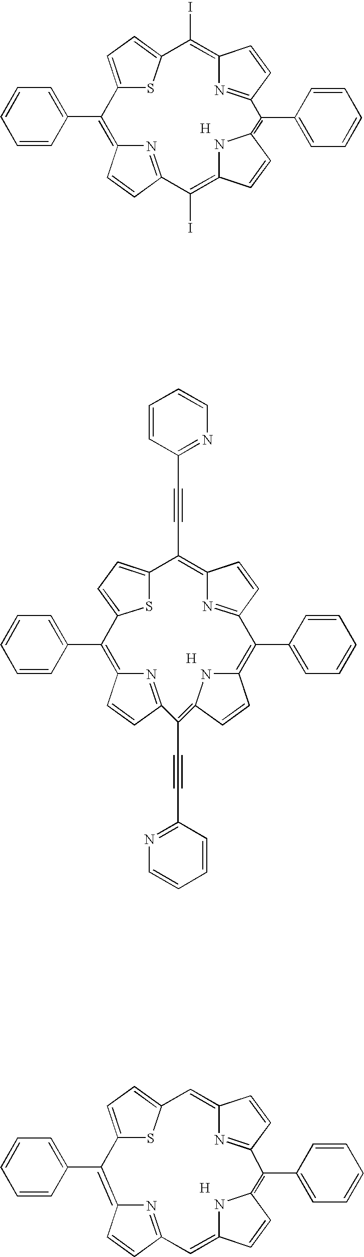

Another preferred embodiment of the invention relates to a compound of formula II wherein

R₁, R₂ and R₃ are the same and are all H, halogen or

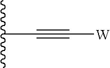

R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl OZ, COOZ, —C≡C—(CH₂)ₚCO₂R₁₀, where R₁₀ is H or alkyl, and O(CH₂)ᵣCOR₁₁, where R₁, is OH, O-alkyl or —N-succinimide, and p and r are each independently an integer from 1 to 10.

In another preferred embodiment of the invention,

R₁, R₂ and R₃ are the same and are all H, halogen or

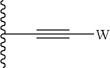

R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl OZ and COOZ.

Another more preferred embodiment of the invention-relates to a compound of formula II wherein

R₁, R₂ and R₃ are the same and are all H, halogen or

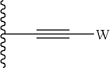

R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl OZ, COOZ, —C≡C—(CH₂)ₚCO₂R₁₀, where R₁₀ is H or alkyl, and O(CH₂)ᵣCOR₁₁, where R₁₁ is OH, O-alkyl or —N-succinimide, and p and r are each independently an integer from 1 to 10.

Preferably, for this embodiment,

R₁, R₂ and R₃ are all H, halogen or

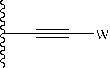

where W is pyridyl or phenyl group, each of which may be optionally substituted by one or more substituents selected from OH, OZ′, and a polyethylene glycol group; and R₄ is a phenyl group substituted by one or more halogen, alkoxy, O(CH₂)ₚCOR₁₁ or —C≡C—(CH₂)ₚCO₂R₁₀ groups.

Even more preferably, the compound of formula If is selected from the following: 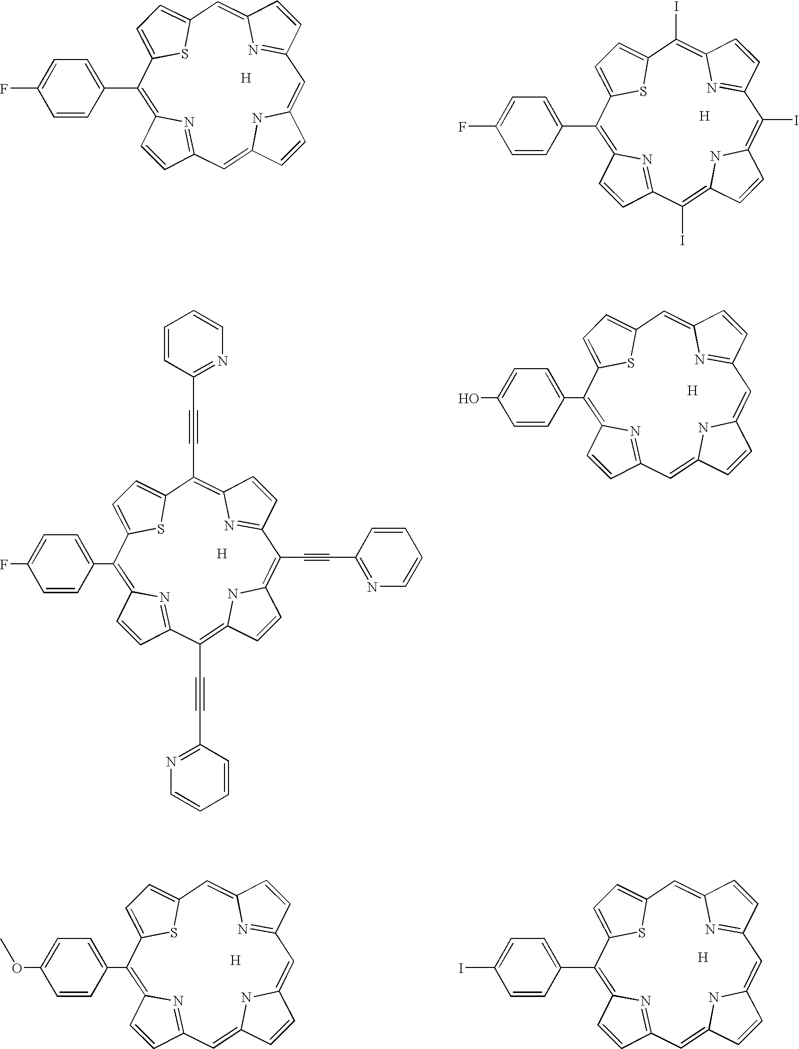 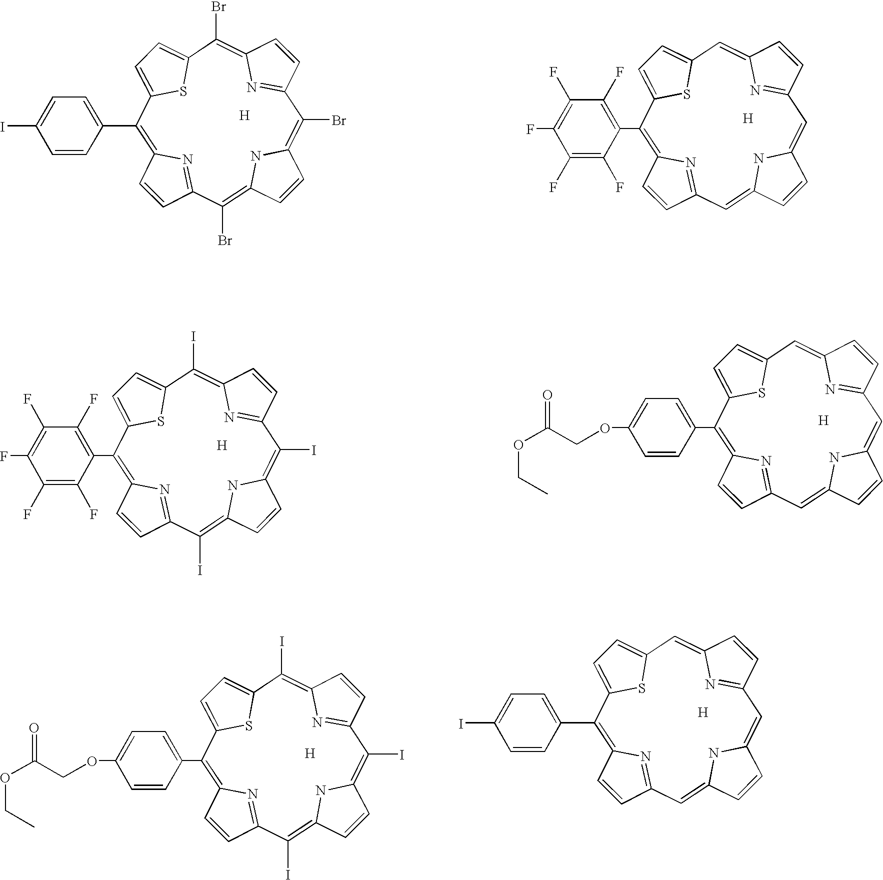 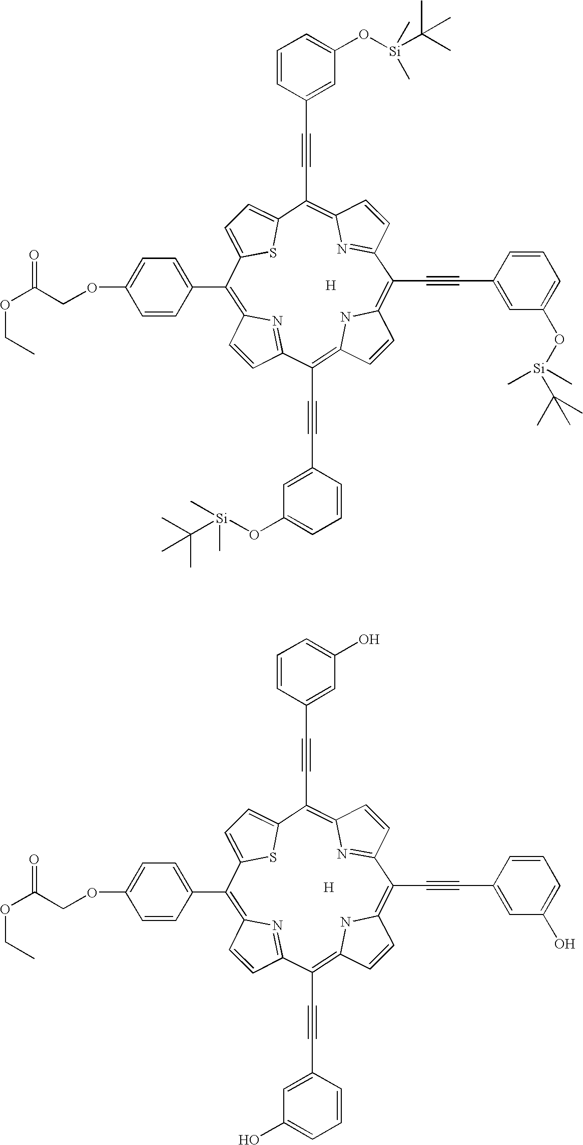 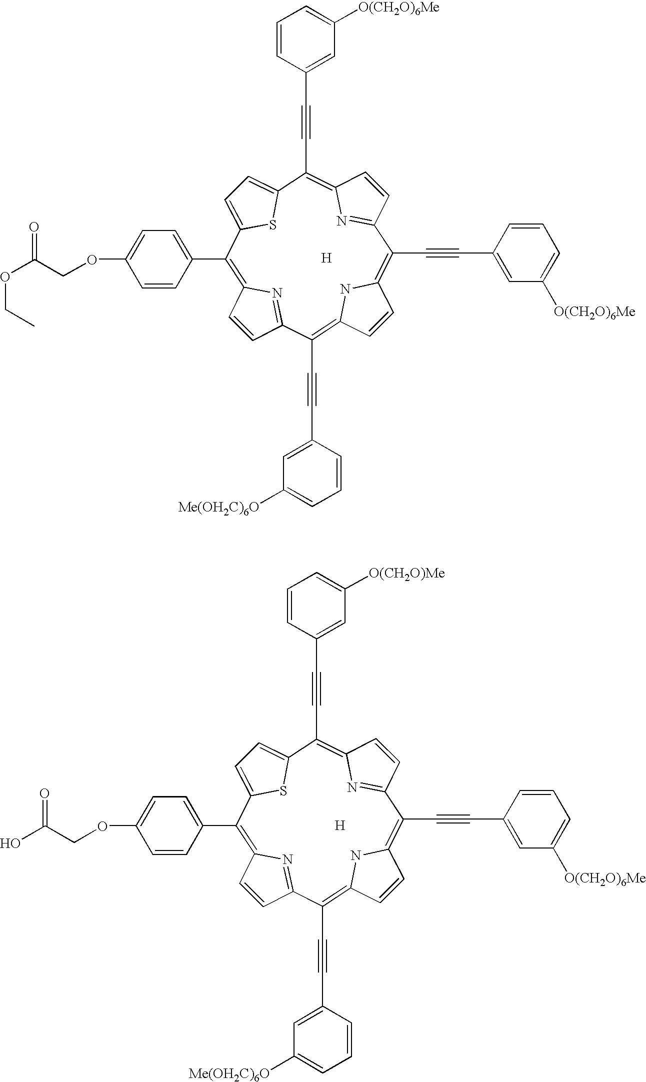 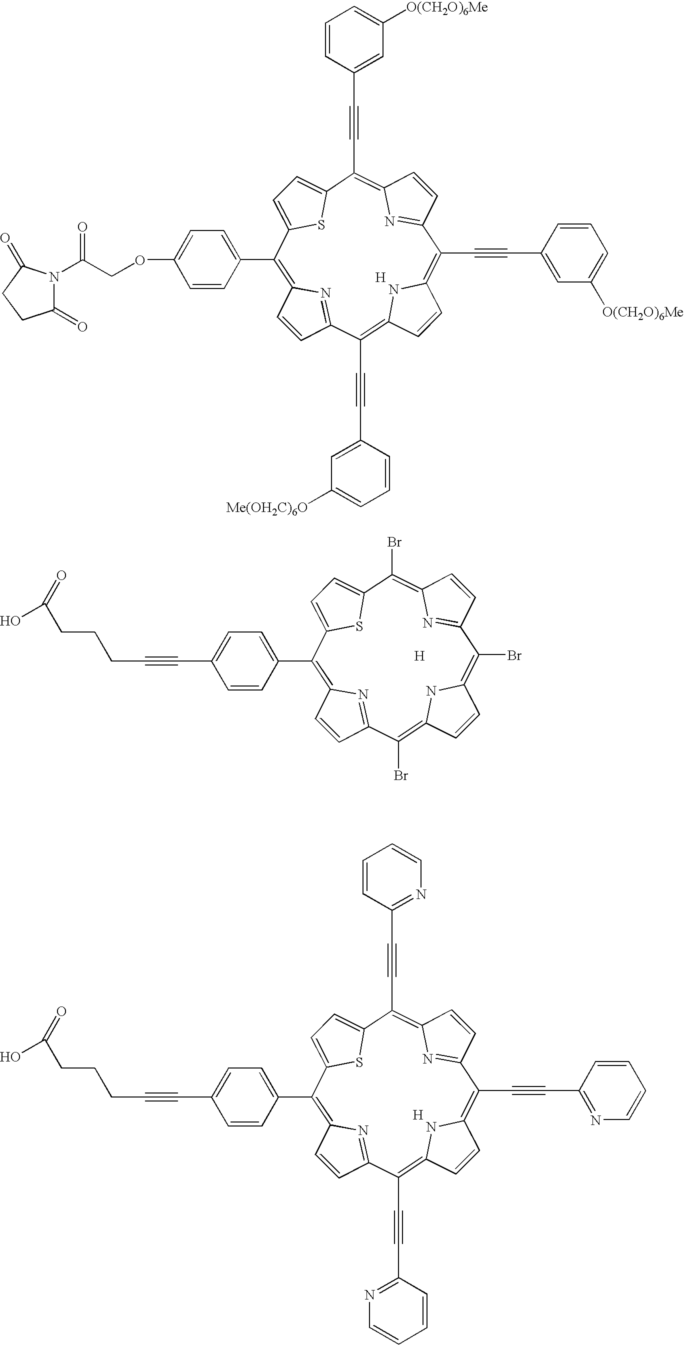
Conjugates

In another aspect, the invention relates to a conjugate molecule comprising a compound according to the invention and a targeting element.

In the context of PDT, fluorescence analysis and imaging, it is highly preferable to exert some degree of control over the localisation of the chromophore in vitro or in vivo. This is particularly important in PDT as the short lifetime of singlet oxygen means that in order to bring about the death of a target cell, the sensitiser must either be positioned immediately alongside or preferably within that cell.

To date, various attempts have been made to control the targeting of porphyrin sensitisers to particular target cells in vivo for the purpose of PDT. Previous efforts at achieving the specific attachment of porphyrin sensitisers to suitable delivery molecules have focused on covalent conjugation to proteins of biological importance, such as human and bovine serum albumins, monoclonal antibodies and lipoproteins. The majority of such bioconjugations have involved chlorin e6, mTHPC (Foscan) or sulphonated phthalocyanines as the sensitiser and have made use of carbodiimide or active ester based methodology. However, the reactive multifinctional nature of these molecules often leads to cross-linkng problems and non-covalent binding.

In a preferred embodiment, the compound of formula I or Ia comprises a haloacetamide group which is capable of cross-linking to the thiol group of a cysteine residue in a protein.

In another preferred embodiment, the compound of formula I or Ia comprises a maleimide group which is capable of cross-linking to the thiol group of a cysteine residue in a protein.

Preferably, the targeting element is selected from a recombinant antibody, a Fab fragment, a F(ab′)₂ fragment, a single chain Fv, a diabody, a disulfide linked Fv, a single antibody domain and a CDRP

As used herein, the term “CDR” or “complementary determining region” refers to the hypervariable regions of an antibody molecule, consisting of three loops from the heavy chain and three from the light chain, that together form the antigen-binding site. By way of example, the antibody may be selected from Herceptin, Rituxan, Theragyn (Pemtumomab), Ifiximab, Zenapex, Panorex, Vitaxin, Protovir, EGFR1 or MFB-23.

In one preferred embodiment, the targeting element is a genetically engineered fragment selected from a Fab fragment, a F(ab′)₂ fragment, a single chain Fv, or any other antibody-derived format.

Conventionally, the term “Fab fragment” refers to a protein fragment obtained (together with Fc and Fc′ fragments) by papain hydrolysis of an immunoglobulin molecule. It consists of one intact light chain linked by a disulfide bond to the N-terminal part of the contiguous heavy chain (the Fd fragment). Two Fab fragments are obtained from each immunoglobulin molecule, each fragment containing one binding site. In the context of the present invention, the Fab fragment may be prepared by gene expression of the relevant DNA sequences. Conventionally, the term “T(ab′)₂”fragment refers to a protein fragment obtained (together with the pFc′ fragment) by pepsin hydrolysis of an immunoglobulin molecule. It consists of that part of the immunoglobulin molecule N-terminal to the site of pepsin attack and contains both Fab fragments held together by disulfide bonds in a short section of the Fc fragment (the hinge region). One F(ab′)₂ fragment is obtained from each immunoglobulin molecule; it contains two antigen binding sites, but not the site for complement fixation. In the context of the present invention, the F(ab′)₂ fragment may be prepared by gene expression of the relevant DNA sequences.

As used herein, the term “Tv fragment” refers to the N-terminal part of the Fab fragment of an immunoglobulin molecule, consisting of the variable portions of one light chain and one heavy chain. Single-chain Fvs (about 30 KDa) are artificial binding molecules derived from whole antibodies, but which contain the minimal part required to recognise antigen.

In another preferred embodiment, the targeting element is a synthetic or natural peptide, a growth factor, a hormone, a peptide ligand, a carbohydrate or a lipid.

The targeting element can be designed or selected from a combinatorial library to bind with high affinity and specificity to the target antigen. Typical affinities are in the 10⁻⁶ to 10⁻¹⁵ M K_{d} range. Functional amino acid residues, present in the targeting element, which could participate in the therapeutic agent attachment reaction may be altered by site-directed mutagenesis where possible, without altering the properties of the targeting element. Examples of such changes include mutating any free surface thiol-containing residues (cysteine) to serines or alanines, altering lysines and arginines to asparagines and histidines, and altering serines to alanines.

The target cells themselves can be human, other mammalian cells or microbial cells (e.g. anti-bacterial PDT using anti-bacterial antibodies [Devanathan, S et al. (1990); PNAS (USA) 87, 2980-2984].

In another preferred embodiment, the conjugate of the invention comprises a polypeptide carrier, a compound according to the invention, and optionally, a targeting element.

In one particularly preferred embodiment, the conjugate comprises a polypeptide carrier and a compound according to the invention. In an especially preferred embodiment, the conjugate comprises a polypeptide carrier which comprises at least one alpha-helix having synthetically attached thereto a compound according to the invention. More preferably still, the conjugate comprises at least one alpha-helix having synthetically attached thereto a plurality of compounds according to the invention, wherein said compounds may be the same or different and are spatially oriented on the polypeptide so as to minimise interactions between said moieties.

As used herein, the term “synthetically attached” encompasses straightforward chemical synthetic techniques and also in vivo synthesis using recombinant DNA techniques.

Preferably, the compounds of the invention are spatially oriented on the polypeptide carrier so as to minimise unfavourable or disruptive interactions between said compounds.

Preferably, the polypeptide carrier of the invention comprises one or more specific amino acid residues for the purpose of site-specific conjugation to said compounds of the invention.

In one preferred embodiment, said specific amino acid residues comprise one or more basic amino acids.

In one preferred embodiment, said specific amino acid residues comprise one or more acidic amino acids.

In another preferred embodiment, said specific amino acid residues comprise one or more hydroxyl-containing amino acids.

In another preferred embodiment, said specific amino acid residues comprise one or more thiol-containing amino acids.

In another preferred embodiment, said specific amino acid residues comprise one or more hydrophobic amino acids. By way of definition, the term “hydrophobic amino acid residue” encompasses amino acids having aliphatic side chains, for example, valine, leucine and isoleucine.

In a particularly preferred embodiment of the invention, the alpha-helix comprises at least two functional amino acid residues positioned so as to protrude externally from said alpha-helix so that each functional amino acid residue does not hinder another. Preferably, the functional amino acid residues are suitable for cross-linking to one or more compounds of the invention. Examples of such functional amino acids include lysine, cysteine, threonine, serine, arginine, glutamate, aspartate, tyrosine.

Typically, the polypeptide may be a conjugate, for example, a protein conjugate, i.e., a fusion protein.

Typically, the a-helix is proteolytically and temperature stable, and is designed so that functional groups from one type of side chain (e.g. basic residues such as lysine and arginine) protrude from the helix in such a way that each functional group is spatially separated from each other.

The length of the helical peptide may be varied to incorporate more or fewer functional amino acid residues, thereby accommodating more or fewer compounds of the invention respectively, as required. Likewise, the position and number of functional amino acid residues can be altered to increase or decrease the distance between the attached photosensitisers, or to vary the number of photosensitisers attached. In each case, the spatial arrangement of the functional amino acid residues is such that there is little or no interference between the photosensitisers attached thereto.

Preferably, the alpha-helix is a 19-residue helix with finctional amino acid residues at positions 2, 8, 10, 14 and 16.

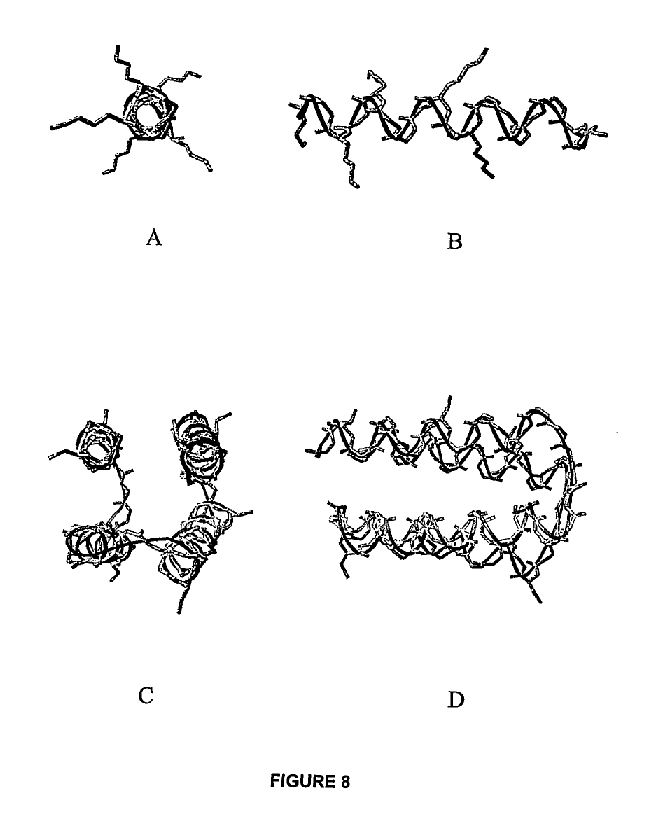

By way of example, and as illustrated in FIG. 8A, the polypeptide carrier may comprise a 19-residue peptide helix with functional amino acids such as lysine or arginine residues at positions 2, 8, 10, 14, 16. This results in an approximately equal number of positively charged residues above/below or either side of the helical axis (viewed in FIG. 8B). These positively charged residues can be seen to be spatially separated when the helix is viewed ‘end on’ (FIG. 8A).

In one preferred embodiment, the polypeptide carrier may comprise two or more alpha-helical polypeptides in the form of a multi-helix bundle. Such multi-helix bundles enable the attachment of a greater number of therapeutic agents. Furthermore, without wishing to be bound by theory, it is believed that multi-helix bundles of this type may exhibit an improved stability over the corresponding single alpha-helical polypeptides.

Thus, in one preferred embodiment, the polypeptide carrier comprises two, three or four alpha-helices, i.e., a two-helix, three helix, or four-helix bundle. Each helix can be of a single-chain or separate chain format.

In one particularly preferred embodiment, the polypeptide carrier further comprises one or more additional amino acid sequences selected from a sub-cellular targeting peptide and a membrane active peptide.

In one preferred embodiment, the sub-cellular targeting peptide targets the nucleus and comprises a sequence selected from KKKKRPR and KRPMNAFIVWSRDQRRK.

In another preferred embodiment, the sub-cellular targeting peptide targets the mitochondria and comprises the sequence MLVHLFRVGIRGGPFP GRLLPPLRFQTFSAVRYSDGYRSSSLLRAVAHLPSQLWA.

In yet another preferred embodiment, the sub-cellular targeting peptide targets lysosomes and comprises the sequence KCPL.

In a ftrther preferred embodiment, the sub-cellular targeting peptide allows proteins to traffic back to the endoplasmic reticulum and comprises the sequence KDEL.

In another preferred embodiment, the membrane active peptide targets the membrane and comprises a sequence selected from the following:

```
    (i) GLFGAIAGFIENGWEGMIDGWYG;
  (ii) GIEDLISEVAQGALTLVP;
(iii) ACYCRIPACIAGERRYGTCIYQGRLWAFDD;
and
  (iv) FFGAVIGTIALGVATSAQITAGIALAEAR.
```

The polypeptide carrier may also comprise a glycosylated protein. For example, the polypeptide may comprise a protein having one or more N- or O-linked carbohydrate residues spatially oriented so as to minimnise interactions between said carbohydrates or compounds of formula I or Ia attached thereto. Thus, in one preferred embodiment, the polypeptide carrier comprises a glycosylated protein (e.g. human serum albumin) or comprises a protein having one or more N- or O-linked glycosylation sites. By way of definition, the term “glycosylated protein” refers to a glycoprotein, i.e., a protein having one or more carbohydrates attached thereto. Typically, glycoproteins contain oligosaccharide units linked to either asparagine side chains by N-glycosidic bonds, or to serine and threonine side chains by O-glycosidic bonds. Accordingly, a protein having N- or O-linked glycosylation sites includes any protein containing amino acid residues having one or more OH or NH₂ side chains.

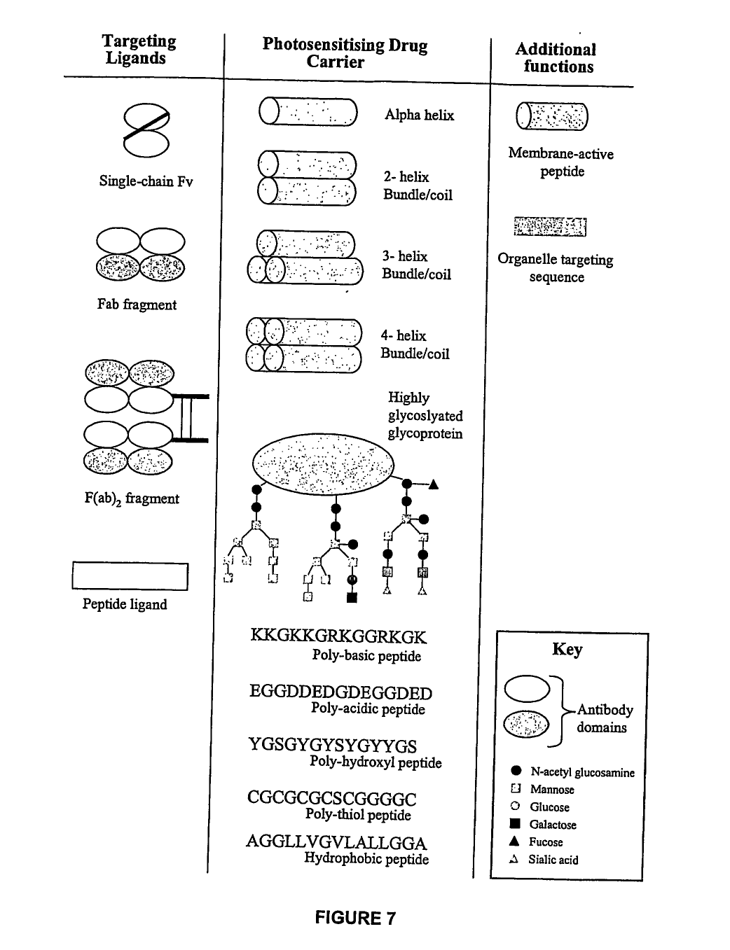

These proteins may be expressed in a eukaryotic system such as mammalian cells, yeasts or insect cells, to ensure full glycosylation. Compounds of the invention whose chemistry is compatible with chemical attachment to hydroxyl or carboxylate groups may be cross-linked onto the glycosylated proteins. The types of carbohydrate residues found on glycosylated proteins are shown in FIG. 7.

In another preferred embodiment of the invention, the polypeptide carrier comprises one or more glycosylation motifs. Typical examples of such glycosylation motifs include Asn-X-Ser and Asn-X-Thr, wherein X is any amino acid residue. Polypeptide sequences including these glycosylation motifs may be expressed in eukaryotic hosts, for example, yeast. Methods for expressing polypeptide sequences may be accomplished by standard procedures well known to those skilled in the art.

After glycosylation, compounds of the invention may be attached to the carbohydrate residues by standard chemical techniques. The spatial arrangement of the glycosylation motifs is such that there is little or no interference between the photosensitiser& attached thereto.

A further aspect relates to the use of a compound of the invention in the preparation of a conjugate as described above.

Pharmaceutical Compositions

Another aspect of the invention relates to a pharmaceutical composition comprising a compound of the invention, or a conjugate thereof as defined above, admixed with a pharmaceutically acceptable diluent, excipient or carrier.

Even though the compounds/conjugates of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The compounds of the present invention can be present as salts or esters, in particular pharmaceutically acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers and tautomers of compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, 35S,¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes solvate forms of the compounds of the present invention. The terms used in the claims encompass these forms.

Polymorphs

The invention furthermore relates to compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10 -1000 mg, preferably between 10 -250 mg, of active ingredient per dose.

Compositions may be formulated in unit dosage form, i.e., in the form of discrete portions containing a unit dose, or a multiple or sub-unit of a unit dose.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

Therapeutic Uses

A further aspect of the invention relates to the use of a compound/conjugate as described hereinbefore in the preparation of a medicament for treating a proliferative disorder.

Preferably, the proliferative disorder is cancer.

As used herein the phrase “preparation of a medicament” includes the use of a compound or conjugate of the invention directly as the medicament in addition to its use in a screening programme for the identification of further agents or in any stage of the manufacture of such a medicament.

Diseases which may be treated according to the invention include cancer, age-related macular degeneration, microbial infections, arthritis and other immune disorders and cardiovascular disease.

Yet another aspect of the invention provides a method of treating a proliferative disorder, said method comprising administering to a subject a therapeutic amount of a compound of the invention, or a conjugate thereof.

Photodynamic Therapy

Another aspect of the invention relates to the use of a compound/conjugate as described hereinbefore in the preparation of a medicament for photodynamic therapy.

Photodynamic therapy (PDT) is a promising new medical treatment that involves the combination of visible light, a drug (photosensitiser) and oxygen to bring about a cytotoxic effect to cancerous or otherwise unwanted tissue. The photosensitiser absorbs light of the appropriate wavelength and undergoes one or more electronic transitions emerging in its excited triplet state. The excited photosensitiser can participate in a one-electron oxidation-reduction reaction (termed Type I) with a neighbouring molecule, producing free radical intermediates that can react with oxygen to produce peroxy radicals and various reactive oxygen species (ROS). Alternatively, the triplet-state photosensitiser can transfer its energy to molecular oxygen (termed Type II) generating singlet molecular oxygen, a highly reactive, powerful and indiscriminate oxidiser that readily reacts with a variety of biological molecules and assemblies. It is generally accepted that singlet oxygen is the primary cytotoxic agent in PDT.

As mentioned, the compounds/conjugates of the present invention may be used as photodynamic therapeutic (PDT) agents. The combination of a sensitiser and electromagnetic radiation for the treatment of cancer is commonly known as photodynamic therapy. In the photodynamic therapy of cancer, dye compounds are administered to a tumour-bearing subject, these dye substances may be taken up, to a certain extent by the tumour. Upon selective irradiation with an appropriate light source (e.g. a laser) the tumour tissue is destroyed via the dye mediated photo-generation of a species such as singlet oxygen or other cytotoxic species such as free radicals, for example hydroxy or superoxide. This requires the sensitiser to have a high triplet yield and lifetime in order to have the best chance of sensitising singlet oxygen production. It also requires a source of laser illumination into the tumour, the cheapest and most penetrating laser light being red.

There are a number of limitations associated with PDT photosensitisers known in the art to date. Firstly, they are often in the form of a complex mixture (as in the case of Photofrin) which makes it difficult to ascertain precisely how the drugs work and how they interacts with tissues in the body. Secondly, photosensitisers such as Photofrin have a tendency to be retained in skin for five to six weeks, inducing undesirable and prolonged photosensitivity under normal daylight. Finally, the longest wavelength of light at which the drugs can be photoactivated is usually well below the wavelength needed for maximum tissue penetration.

The compounds/conjugates of the present invention are advantageous in the context of PDT as they typically possess one or more of the following characteristics:

(i) they are chemically pure and of known (constant) composition;

(ii) they have minimal dark toxicity and only be cytotoxic in the presence of light;

(iii) they have a strong absorption at longer wavelengths (between 650-800 nm) when tissue penetration of light is at a maximum, while still being energetic enough to produce singlet oxygen;

(iv) they exhibit a high quantum yield for singlet oxygen;

(v) they are rapidly excreted from the body, thereby inducing low systemic toxicity;

(vi) they are preferentially retained by the target tissue;

(vii) they are water soluble and easy to formulate (to aid delivery of the drug), and stable to avoid the formation of metabolites.

Phosphoimmunoassays

Yet another aspect of the invention relates to the use of compounds or conjugates of the invention in phosphoimmunoassays (PIA) and/or in the measurement of dissolved oxygen levels in biological systems.

It known in the art that metalloporphyrins, particularly the Pt and Pd complexes, are potentially useful in phosphoimmunoassays (PIA) [A P Savitsby et al, Dokl. Acad. Nauk SSSR, 1989, 304, 1005]. Furthermore, the extreme sensitivity of the triplet excited states of these metalloporphyrins to dissolved oxygen has been used to measure dissolved O₂ levels in biological systems [T J Green et al, Anal. Biochem., 1988, 174, 73; EP 0127797A and U.S. Pat. No. 4,707,454]. Since the compounds of the present invention exhibit very high triplet yields, they are expected to exhibit improved characteristics with respect to PIA and/or sensitivity to dissolved O₂.

Medical Imaging

One aspect of the invention relates to the use of a compound or conjugate as described hereinbefore for medical imaging. By way of example, water soluble paramagnetic manganese complexes of the compounds of the invention, including Mn(III) complexes, may be used in methods for enhancing images obtained from magnetic resonance imaging of a region of regions containing a malignant tumour growth.

Other Applications

The compounds of the present invention may be used in a broad range of other applications. By way of example, these may include use as pigments or dyes, as components of discotic liquid crystal phases (in particular they may also be used a precursors for discotic liquid crystals), as two dimensional conjugated polymeric arrays [Drain and Lehn, J. Chem. Soc., Chem. Commun., 1994, 2313]; as reverse saturable absorbers and as molecular wires (R. J. M. Nolte at al, Angew. Chem. Int. Ed. Eng., 1994, 33(21), 2173). The types of liquid crystal devices include linear and non-linear electrical, optical and electro-optical devices, magneto-optical devices and devices providing responses to stimuli such as temperature changes and total or partial pressure changes. The compounds of the present invention may also be used in biaxial nematic devices and as second or third order non-linear optic (NLO) materials.

The compounds of the present invention may be suitable as optical storage media and may be combined with dyes for use in laser addressed systems, for example in optical recording media. Typically the compound will absorb in the near-infrared. In order to make an optical recording media using a near-infrared absorber, the near-infrared absorber may be coated or vacuum-deposited onto a transparent substrate. EP 0337209 A2 describes the processes by which the above optical-recording media may be made. The compounds of the present invention are also useful in near-infrared absorption filters and liquid crystal display devices. As described in EP 0337209 A2, display materials can be made by mixing a near-infrared absorber of the invention with liquid crystal materials such as nematic liquid crystals, smectic liquid crystals and cholesteric liquid crystals. The compounds of the present invention may be incorporated into liquid crystal panels wherein the near infrared-absorber is incorporated with the liquid crystal and a laser beam is used to write an image. Mixtures of compounds of the current invention may be mixed with liquid crystal materials in order to be used in guest-host systems. GB 2,229,190 B describes the use of phthalocyanines incorporated into liquid crystal materials and their subsequent use in electro-optical devices.

It may also be advantageous to polymerise certain of the compounds of the current invention. There are numerous ways in which the compounds may be incorporated into a polymer. Polymerisation may be effected by one or more of the positions R₁-R₄ in formula I or Ia, or via the central metal atom or metal compound, or by a combination of the above techniques.

Polymerised compounds may also be used in Langmuir Blodgett films. Langmuir Blodgett films incorporating compounds of the present invention may be laid down using conventional and well known techniques, see R. H. Tredgold in “Order in Thin Organic Films”, Cambridge University Press, p 74, 1994 and reference therein. Langrnuir Blodgett Films incorporating compounds of the present invention may be used as optical or thermally addressable storage media.

Synthesis

The present invention also provides a more flexible and improved synthetic strategy for the synthesis of core-modified porphyrins, and reduced derivatives thereof, in which the number of meso substiuents around the macrocycle periphery can be varied from 1 to 4. The “freeing up” of the meso positions around the macrocycle core allows further synthetic manipulation to be carried out in order to fine tune the absorption and photophysical properties, and to incorporate fimctional groups which allow specific attachment to biological carriers.

Reaction schemes showing the synthetic strategy for preparing selected compounds of the invention are shown in FIGS. 11-15.

A further aspect of the invention relates to a process for preparing a compound of formula I or Ia as defined above, said process comprising reacting a compound of formula VII with a dipyrrole to form a compound of formula IX

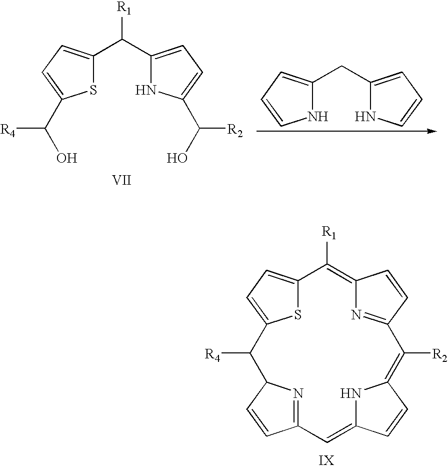

where R₁, R₂ and R₄ are as defined above.

Preferably, said compound of formula VII is prepared via intermediates X, XI and XII

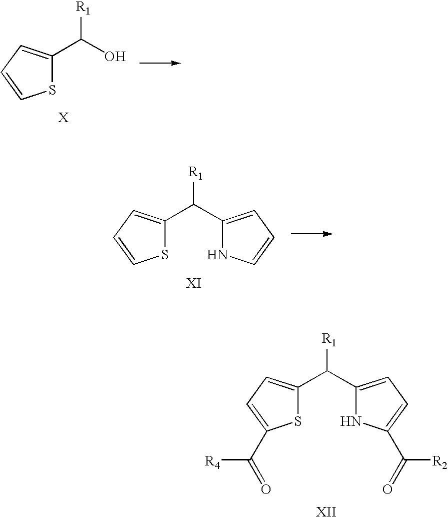

In another preferred embodiment, the invention provides a process for preparing compounds of formula III, IV, V or VI, which flirther comprises oxidising said compound of formula VII with osmium tetroxide.

The present invention is further described by way of example, and with reference to the following figures wherein:

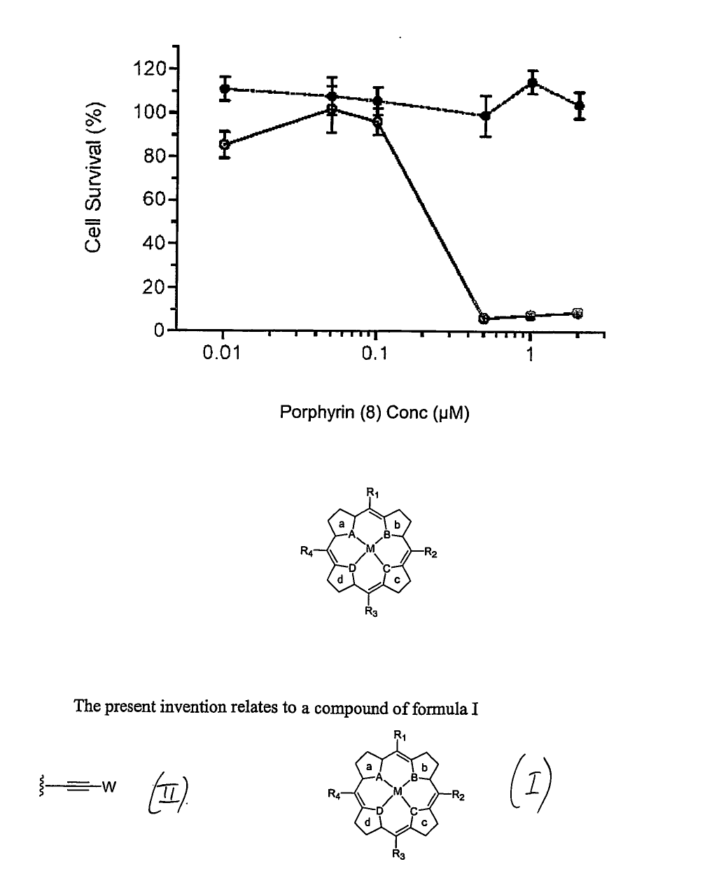

FIG. 1 shows the photodynamic activity of porphyrin (8). The filled circles and dotted line shows the cell survival observed with 0.01 μM to 2 μM porphyrin (8) in the absence of light. The open circles and solid line show the cell survival observed with the same concentrations of porphyrin (8) irradiated with 100 J cm⁻² white light. Each point represents the mean of 6 replicate incubations.

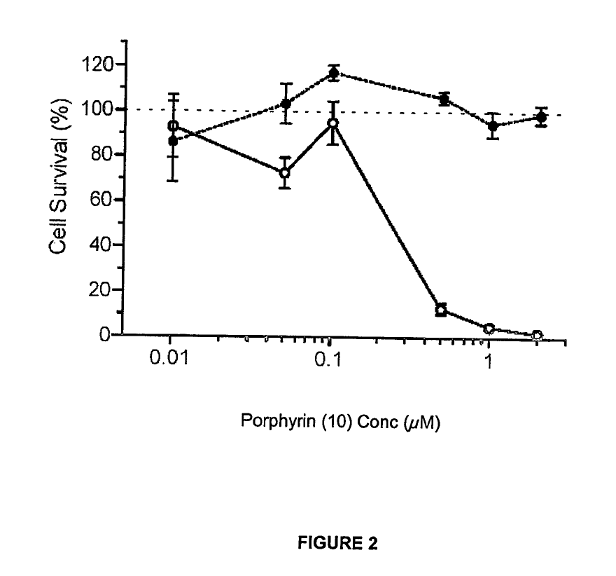

FIG. 2 shows the photodynamic activity of porphyrin (10). The filled circles and dotted line shows the cell survival observed with 0.01 μM to 2 μM porphryin (10) in the absence of light. The open circles and solid line show the cell survival observed with the same concentrations of porphyrin (10) irradiaited with 100 J cm⁻² white light. Each point represents the mean of 6 replicate incubations.

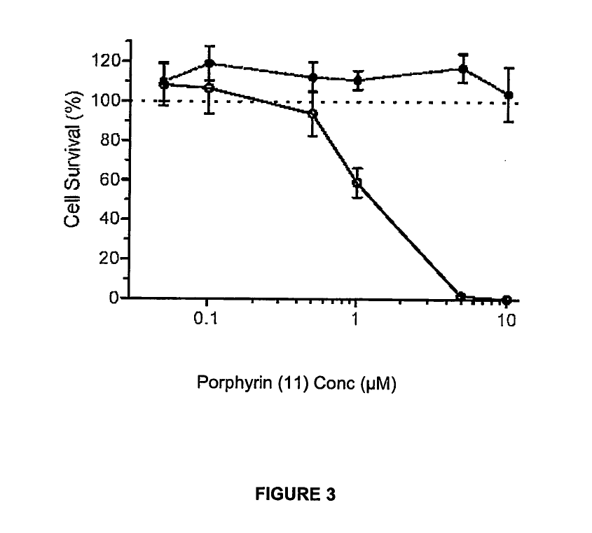

FIG. 3 shows the photodynamic activity of porphyrin (11). The filed circles and dotted line shows the cell survival observed with 0.05 μM to 10 μM porphyrin (11) in the absence of light. The open circles and solid line show the cell survival observed with the same concentrations of porphyrin (11) irradiated with 100 J cm⁻² white light. Each point represents the mean of 6 replicate incubations.

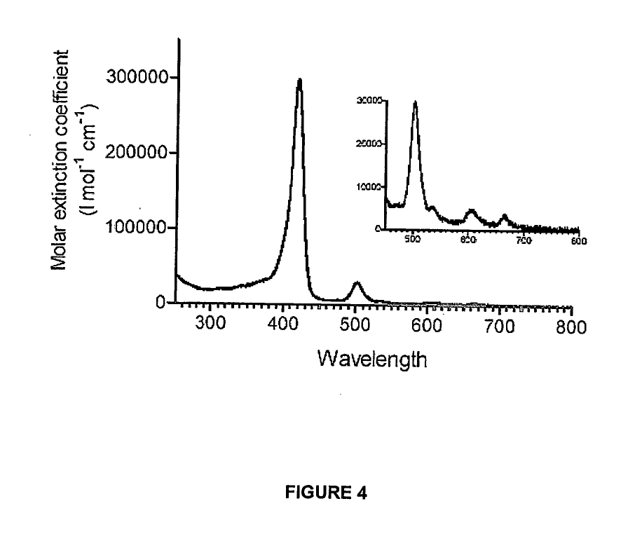

FIG. 4 shows the molar extinction spectrum of porphyrin (8) in THF.

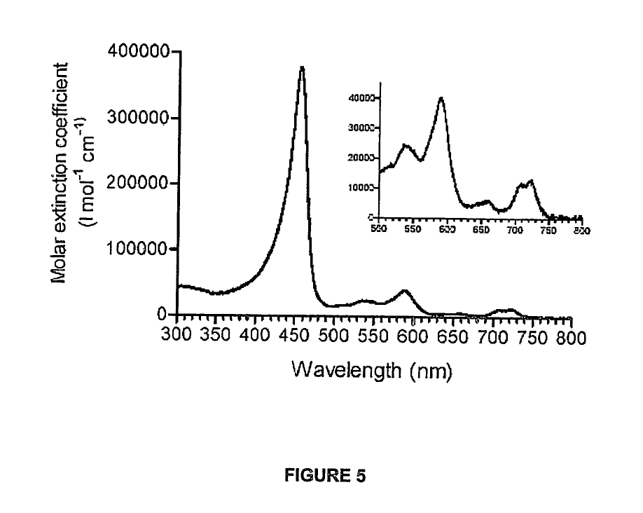

FIG. 5 shows the molar extinction spectrum of porphyrin (10) in THF.

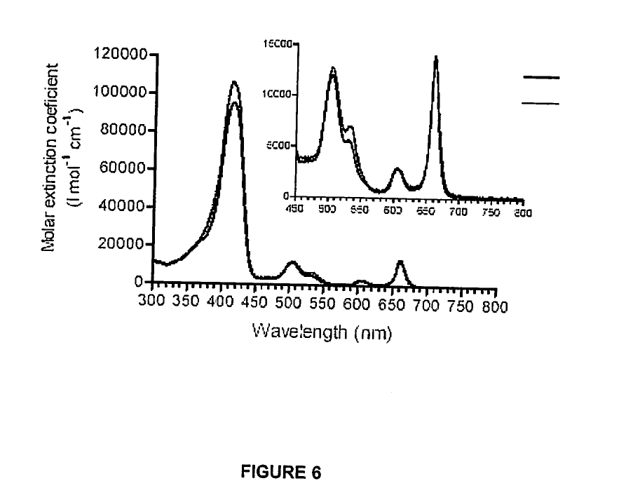

FIG. 6 shows the molar extinction spectra of porphyrin (11) and (12) in THF [solid line porphyrin (11) and dashed line porphyrin (12)].

FIG. 7 shows the modular structure of the multifunctional targetable-carrier protein of the invention.

FIG. 8 shows the molecular structure of helical based carrier proteins for the compounds of the present invention. In more detail, FIGS. 8(A) and (B) show a single peptide cc-helix engineered to contain optimally-spaced lysine or arginine residues, which can be used to deliver compounds. Side (13) and end-on (A) views show favourable spacing of the amino groups used to attach the compounds. FIGS. 8(C) and (D) show a 4-helix bundle, engineered to contain optinally-spaced cysteine residues, which can be used to deliver compounds. Side (B) and end-on (A) views show favourable spacing of the thiol groups used to attach the compounds.

FIG. 9 shows shows the construction of an scFvhelix bundle fusion gene. In more detail, FIG. 9 shows how a scFv and a 4-helix bundle gene would be assembled in a bacterial expression vector to produce the scFv-helix bundle fusion protein.

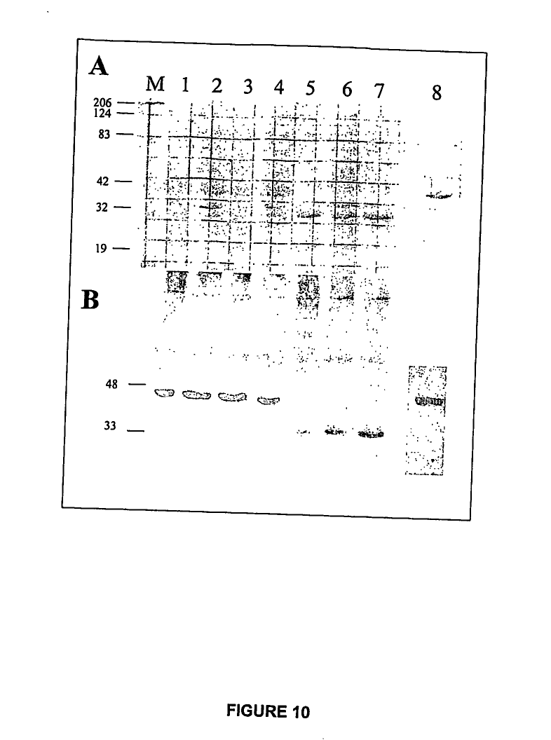

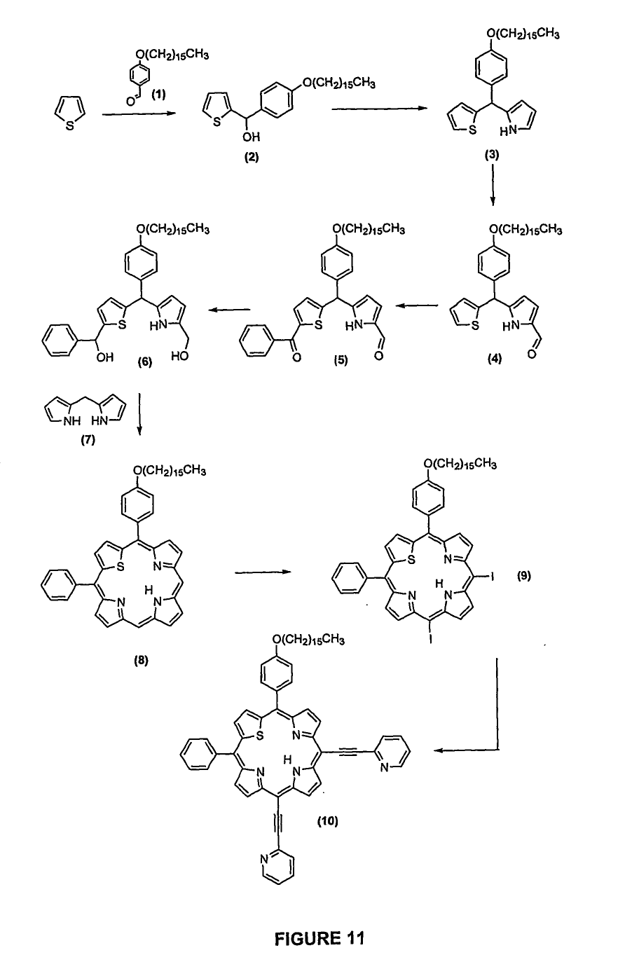

FIG. 10 shows over-expression anti-CEA scFv (lanes 5-7) and scFv-4 helix bundle (lanes 1-4) fusion protein in E. coli BL21(DE3). (A) Whole cell lysates are analysed by SDS-PAGE stained with coomassie blue. (B) Whole cell lysates are analysed by western blot using a mouse anti-His tag monoclonal antibody (Qiagen) followed by anti mouse-horseradish peroxidase (Sigma) developed by ECL (Amersham). M-molecular weight markers in KDa. Lane 8 represents substantially pure scFv-4 helix bundle fusion protein after IMAC on Nickel sepharose.

FIG. 11 shows the synthetic route to core-modified porphyrins in which R₂=R₃; R₁≠R₄; and R₁, R₄≠R₂, R₃.

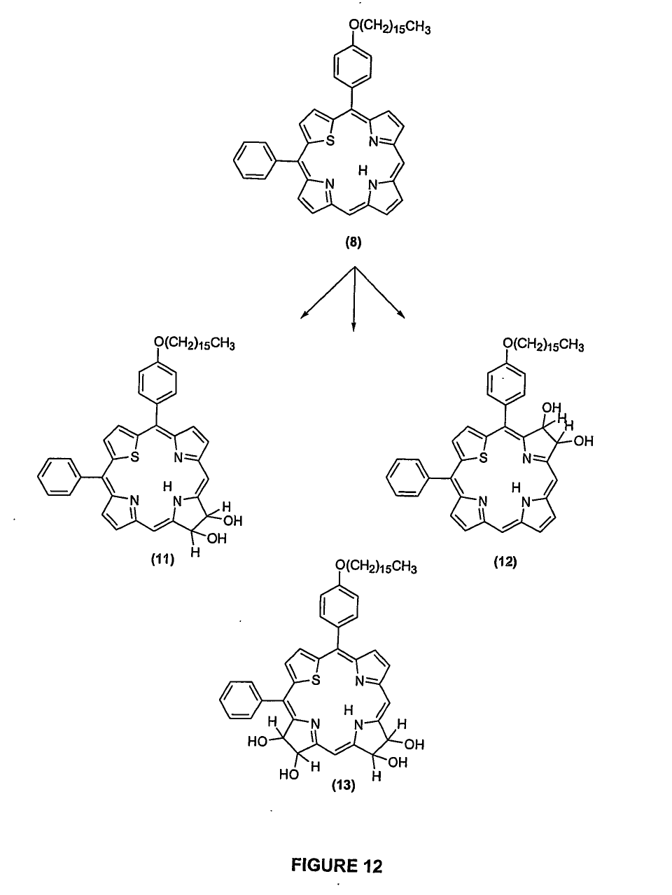

FIG. 12 shows the synthetic route to core-modified chlorins and isobacteriochlorins in which R₂=R₃; R₁≠R_{4;} and R₁,R₄≠R₂, R₃.

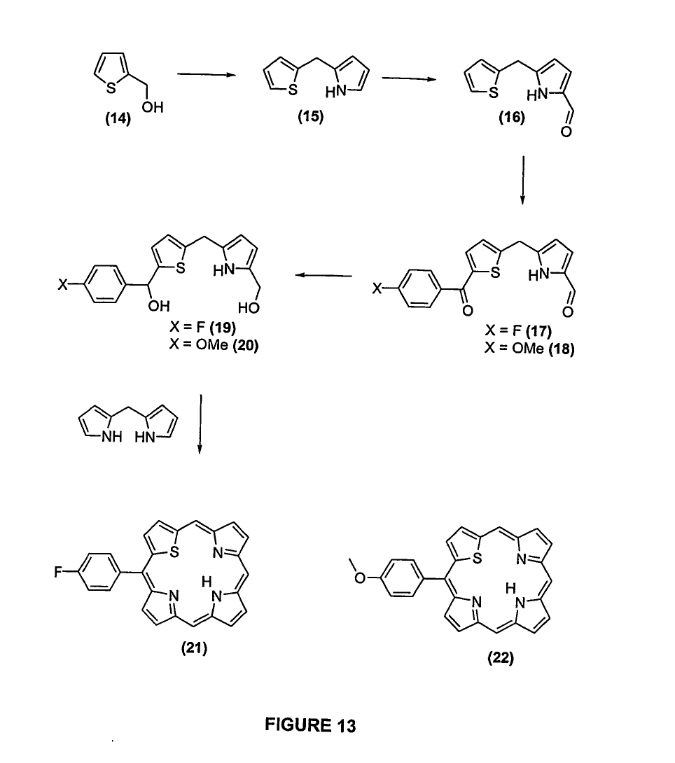

FIG. 13 shows the synthetic route to core-modified porphyrins in which R₁, R₂ and R₃ are identical, and R₄≠R₁, R₂, R₃.

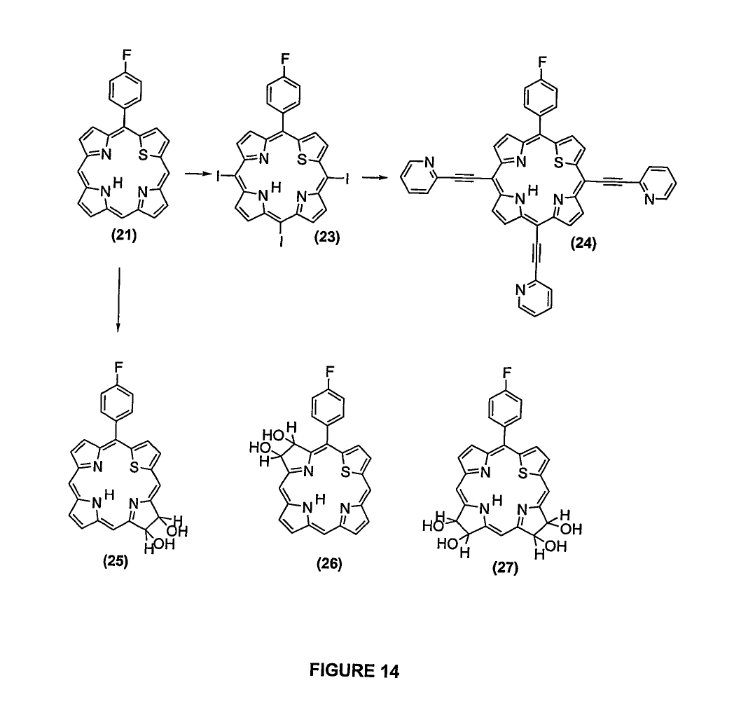

FIG. 14 shows the synthetic route to further core-modified porphyrins, and the corresponding chlorins and isobacteriochlorins, in which R₁, R₂ and R₃ are identical, and R₄≠R₁, R₂, R₃.

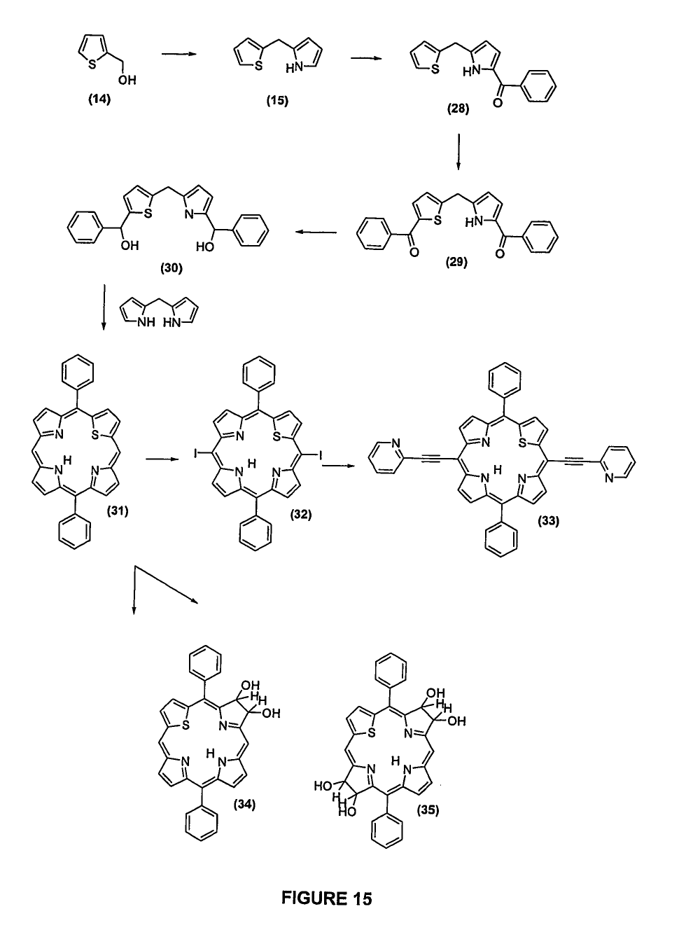

FIG. 15 shows the synthetic route to core-modified porphyrins, and the corresponding chlorins and bacteriochlorins, in which R₁=R₃; R₂=R₄, where R₁, R₃≠R₂, R₄.

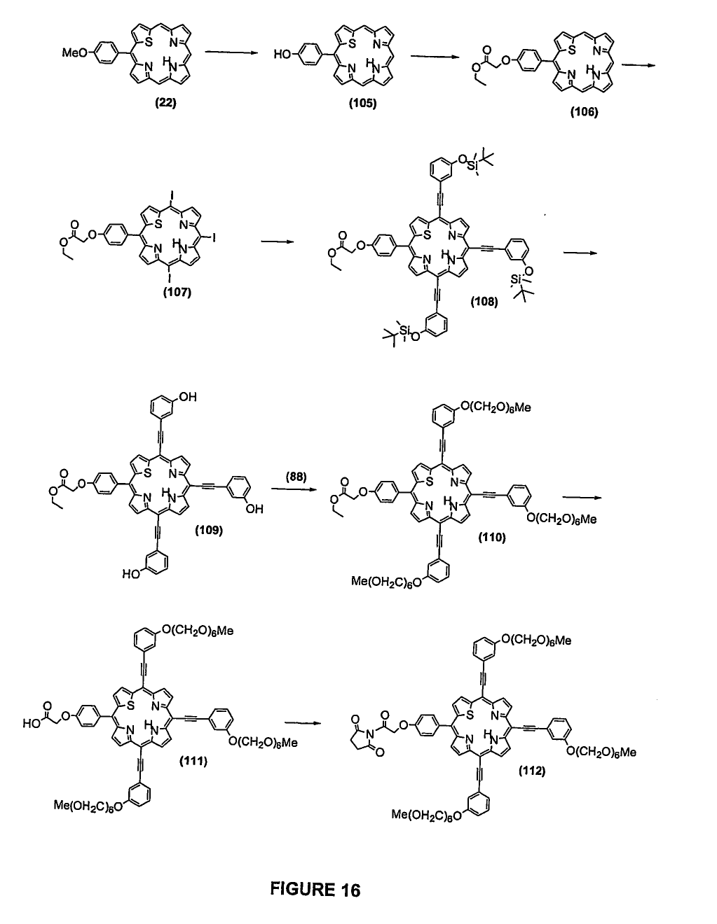

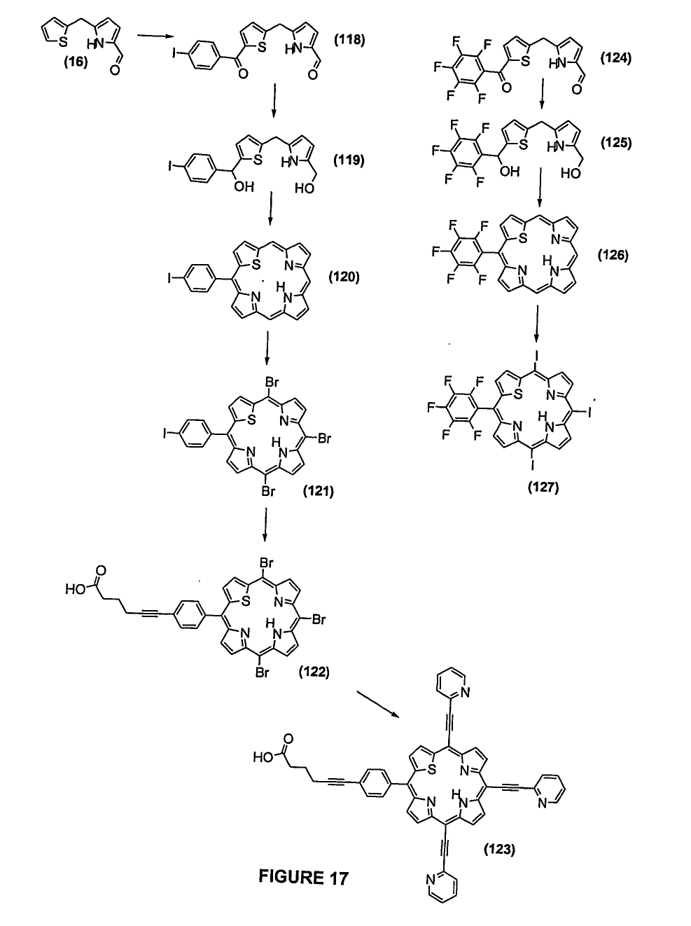

FIGS. 16 and 17 show the synthetic routes to further core-modified porphyrins in which R₁, R₂ and R₃ are identical, and R₄≠R₁, R₂, R₃.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### EXAMPLES

### 4-Hexadecyloxybenzaldehyde [1]

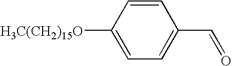

To a solution of 4-hydroxybenzaldehyde (10 g, 0.082 mol), in dry DMF (100 ml) anhydrous K₂CO₃ (34 g, 0.246 mol) was added and the solution stirred for a few minutes before the addition of 1-Bromohexadecane (32.5 g, 0.107 mol) as a neat liquid. The reaction mixture was stirred for a further 6 h at 60-70° C. under argon. On completion the mixture was allowed to cool to room temperature then poured into a large volume of ice/water (800 ml). The aldehyde was extracted with diethyl ether (3×150 ml) and the combined extracts washed well with water (5×200 ml) and brine (2×150ml), dried over Na₂SO₄, filtered and the solvent was removed to give a semi-solid. This was stirred with ice-cold methanol and filtered to give the desired aldehyde as a white solid (27.3 g, 96%), mp: 64-65° C. ¹H NMR δ (ppm, CDCl₃) 9.87 (s, 1H, —CHO); 7.82 (d, 2H, Ar—H, J=8.71 Hz); 6.98 (d, 2H, Ar—H, J=8.71 Hz); 4.034 (t, 2H, OCH2 J=6.6 Hz); 1.87-1.77 (m, 2H, OCH₂CH₂CH₂); 1.48-1.42 (m, 2H, CH₂CH₂CH₃); 1.26 (m, 24H, —CH₂); 0.877, (t, 3H, —CH₃ ). ¹³C NMR δ (ppm, CDCl₃) 191.25 (—CHO); 164.68, 132.40, 130.13, 115.15 (Ar—C); 68.84 (—OCH₂), 34.50-23.11 (—CH₂, 14C); 14.54 (—CH₃). MS (EI⁺) m/z 346 (M⁺, 40%), 123 (80), 43 (100); found 346.286 (calculated for C₂₃H₃₈O₂ 346.287)

### (4-Hexadecyloxy-phenyl)-thiophel-2-yl-methanol [2]

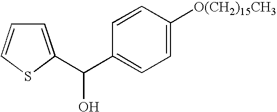 Ref: J. Org. Chem., 1999, 64(2), 7891

Thiophene (2 ml, 25.98 mmol) was added to a solution of TMEDA (5.84 ml, 38.72 mmol) and butyl lithium (1.6M solution in hexanes) (17.1 ml, 27.48 mmol) in anhyrous hexane (70 ml). The mixture was refluxed for 30 min. then allowed to cool slightly after which it was introduced directly using a canula into an ice-cold solution of the benzaldehyde (1) (8.8 g, 25.46 mmol) in anhydrous hexane (small amount of dry TBF was added to aid the dissolution of the aldehyde). Once addition was complete, the mixture was stirred for an additional 30 min. at room temperature, it was then quenched by the addition of a saturated solution of ammonium chloride and extracted with diethyl ether (2×300 ml). The combined ether extracts was dried with anhydrous sodium sulphate, filtered and evaporated to give the desired compound as a creamy-white solid ¹H NMR δ (ppm, CDCl₃) 7.33 (d, 2H, J=7 Hz, Ar—H), 7.23 (dd, 1H, J=3.7 Hz and J=1.2 Hz, thiophene-H), 6.92 (m, 1H, thiophene-H), 6.86 (m, 3H, thiophene-H and Ar—H), 5.99 (s, 1H, C—H), 3.93 (t, 2H, 6.7 Hz, O—CH₂), 1.75 (m, 2H, —CH₂), 1.24 (m, 26H, —CH₂), 0.88 (t, 3H, J=7 Hz); MS (EI⁺) m/z 430 (M⁺)

### 2-[(4-hexadecyloxy-phetyl)-thiphen-2-yl-methlyl]-1H-pyrrole [3]

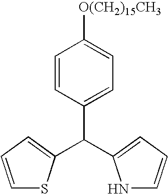 Ref: J. Org. Chem., 1999, 64(2), 7891

A solution of compound (3) (2 g, 4.65 mmol) in pyrrole (7.53 ml, 0.11 mol) was degassed with argon for approx. 5 min. after which BF₃.OEt₂ (0.58 ml, 5.81 mmol) was added and the reaction stirred at room temperature for 30 min. before being quenched by the addition of 0.1M sodium hydroxide solution. The mixture was extracted with ethyl acetate (2×200 ml), the combined extracts were then washed with water followed by brine before being dried over anhydrous magnesium sulphate. Evaporation of the solvent gave a brown sticky oil, this was placed on a high vacuum pump to remove the last traces pyrrole and the residue stirred with cold methanol to give the desired pyrromethane as a white solid 1.72 g (78%). ¹H NMR δ (ppm, CDCl₃) 7.90 (br s, 1H, NH), 7.18 (dd, 1H, J=3.7 Hz and 1.2 Hz, thiophene-H), 7.14 (d, 2H, J=7 Hz, Ar—H), 6.92 (m, 1H, thiophene-H), 6.82 (d, 2H, J=7 Hz), 6.79 (m, 1H, thiophene-H), 6.68 (m, 1H, pyrrole-H), 6.14 (q, 1H, J=2.7 Hz and J=6.2 Hz, pyrrole-H), 5.6 (s, 1H, C—H), 3.91 (t, 2H, 6.7 Hz, O—CH₂), 1.76 (m, 2H, —CH₂), 1.25 (m, 26H, —CH₂), 0.89 (m, 3H, —CH₃); MS (FAB⁺) m/z 479 (M⁺, 90%), 413 (100), 189 (82), 162 (76); found 479.32252 (calculated for C₃₁H₄₅NOS 479.322187)

### 2-[(4-hexadecyloxy-phenyl)-thiphen-2-yl-methyl]-1H-pyrrole-2-carbeldehyde [4]

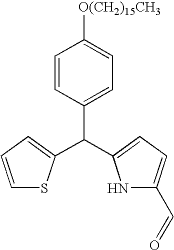

Vilsmeir reagent was prepared by the dropwise addition of POCl₃ (1.5 ml) to a stirred solution of anhydrous DMF (10 ml) under nitrogen at 0° C. The resulting light-yellow solution was stirred for 15 min. prior to use.

To a stirred solution of the pyrromethane (3) (1.35 g, 2.81 mmol) dissolved in anhydrous DMF (10 ml) at 0° C. and under nitrogen, the Vilsmeir reagent (3 ml) was added dropwise. The mixture stirred for 2 h and quenched by the addition of 1M sodium hydroxide solution (100 ml), the resulting dark mixture was extracted with ethyl acetate (3×50 ml), the combined organic extracts washed with water (3×50 ml) then brine (100 ml) before being dried (MgSO₄) and concentrated in vacuo to give a pale brown oil. Purification by column chromatography [silica gel: 30% EtOAc/hexanes] gave the desired mono-formyl pyrromethane 1.12 g (79%). ¹H NMR δ (ppm, CDCl₃) 9.4 (s, 1H, CHO), 9.08 (br, 1H, NH), 7.21 (dd, 1H, J=5 Hz), 7.12 (d, 2H, J=7 Hz, Ar—H), 6.94 (m, 1H, thiophene-H), 6.89 (m, 1H, pyrrole-H), 6.84 (d, 2H, J=7 Hz, Ar—H), 6.79 (m, 1H, thiophene-H), 6.1 (m, 1H, pyrrole-H), 5.6 (s, 1H, C—H), 3.9 (t, 2H, J=6 Hz, O—CH₂), 1.2-1.8 (m, 28H, —CH₂), 0.89 (t, 3H,J=7 Hz, CH₃); MS (EI⁺) m/z 507 (M⁺, 100%), 478 (25), 254 (20); found 507.317851 (calculated for C₃₂H₄₅NO₂S 507.371702)

### 5-[5-Benzoyl-thiophen-2-yl)-(4-hexadecyloxy-phenyl)-methyl]-1H-pyrrole-2-carbaldehyde [5]

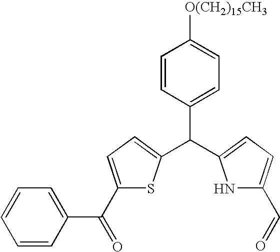

A solution of pyrromethane (4) (0.263 g, 0.52 mmol) in anhydrous DCM was added to a stirred ice-cold mixture of benzoyl chloride (140 μl, 1.19 mmol) and powdered aluminium chloride (0.179 g, 1034 mmol) in anhydrous DCM (40 ml). The mixture was stirred at room temperature under nitrogen for 4 h before being concentrated and the residue purified by column chromatography (silica gel: 30% EtOAc/hexanes) to give the desired pyrromethane 0.158 g (49%). ¹H NMR δ (ppm, CDCl₃) 9.8 (br, 1H, NH), 9.35 (s, 1H, CHO), 7.8 (m, 2H, Ar—H), 7.54 (m, 1H, thiophene-H), 7.46 (m, 3H, Ar—H), 7.13 (d, 2H, J=7 Hz, Ar—H), 6.8-6.9 (m, 4H, thiophene-H, Ar—H and pyrrole-H), 6.1 (m, 1H, pyrrole-H), 5.7 (s, 1H, C—H), 3.9 (t, 2H, J=7 Hz, O—CH₂), 1.75 (m, 2H, —CH₂), 1.2-1.6 (m, 26H, —CH₂), 0.89 (t, 3H, J=7 Hz, —CH₃); MS (EI⁺) m/z 611 (M⁺), 425, 350; found 611.344273 (calculated for C₃₉H₄₉NO₃S 611.343317)

### {5-[(4-Hexadecyloxy-phenyl)-(5-hydroxymethyl)-1H-pyrrol-2-yl)-methyl]-tlziopheti-2-yl}-phenylmetlhanol [6]

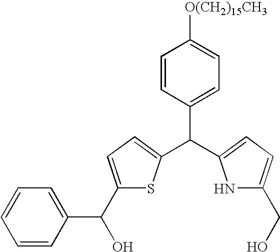

To a stirred solution of pyrromethane (5) (0.342 g, 0.56 mmol) in THF/MeOH (1:1, 50 ml) sodium borohydride (2012 g, 56 mmol) was added portion wise, taking care to control the effervescence. The reaction was stirred for 30 min. at room temperature, when TLC (silica gel: 30% EtOAc/hexanes) showed the consumption of starting material and the presence of a single new product spot. The reaction mixture was quenched by the addition of water (100 ml, CARE!), and extracted with DCM (3⇄50 ml). The combined organic extracts were dried over anhydrous potassium carbonate and concentrated to give an oil in quantitative yield. This was used immediately and without further purification.

### 5-(4-hexadecyloxyphenyl)-10-(phenyl)-15,20-H-21-thiaporphryin [8]

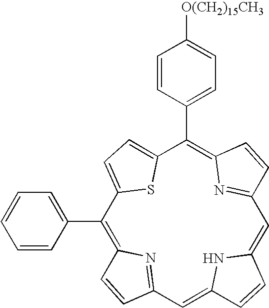

To a stirred solution of the dipyrromethanediol (7) (0.35 g, 0.56 mmol) and dipyrromethane (9) (82 mg, 0.56 mmol) in acetonitrile (50 ml) at 0° C., ammonium chloride (0.299 g, 5.6 mmol) was added followed by BF₃. Et₂ (67 μl, 0.56 mmol). The mixture was stirred under nitrogen, at room temperature and in the dark for 25 min., DDQ (254 mg, 1.12 mmol) was added and the reaction mixture stirred for a further 1 h open to the air. Triethyl amine (2 ml) was then added and the mixture stirred for a furter 1 h after which the reaction mixture was passed through a large pad of silica gel, eluting with DCM until the washings were clear. The filtrate was concentrated and purified by column chromatography [silica gel: 80% DCM/hexanes] to give the desired porphyrin 39 mg (10%) ¹H NMR δ (ppm, CDCl₃) 10.25 (s, 1H, meso-H), 10.24 (s, 1H, meso-H), 9.88 (d, 1H, J=5 Hz, thiophene-H), 9.83 (d, 1H, J=5 Hz, thiophene-H), 9.55 (s, 2H, pyrrole-H), 9.18 (d, 2H, J=6 Hz, pyrrole-H), 8.92 (d, 1H, J=5 Hz, pyrrole-H), 8.88 (d, 1H, J=5 Hz, pyrrole-H), 8.28 (m, 2H, Ar—H), 8.18 (m, 2H, Ar—H), 7.84 (m, 3H, Ar—H), 7.36 (d, 2H, J=9 Hz, Ar—H), 4.25 (t, 2H, J=7 Hz, O—CH₂), 2.0 (m, 2H, —CH₂), 1.3-1.7 (m, 27H, —CH₂), 0.89 (t, 3H, J=7 Hz, —CH₃), −3.4 (s, 1H, NH); MS (FAB⁺) m/z 720 (M⁺, 100%), 508, 495, found 720.401947 (calculated for C₄₈H₅₄N₃OS 720.39876); UV-Vis (DCM) λ₍ₘₐₓ₎ 418, 502, 532, 606, 668 nm

### 5-(4-hexadecyloxyphenyl)-10-(phenyl)-15,20-diiodo-21-thiaporphryin [9]

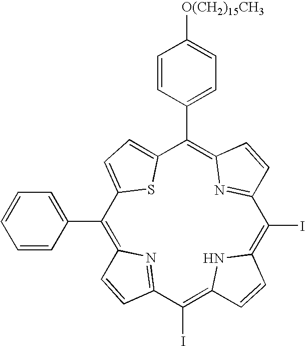

To a light protected solution of (CF₃CO₂)₂PhI (51 mg, 0.12 mmol) in dry CHCl₃ (10 ml) a solution of iodine (25 mg, 0.1 mmol) in dry chloroform (5 ml) was added followed by a few drops of pyridine. The resulting mixture was stirred at room temperature under nitrogen until a pale yellow solution formed. This solution was then added drop wise to a light protected solution of porphyrin (8) (36 mg, 50 μmol) in anhydrous chloroform (25 ml). The addition took 25 min. after which the mixture was stirred for a further 14 h under nitrogen. The reaction mixture was then diluted with chloroform (100 ml) and washed with saturated sodium thiosulphate solution (2×100 ml). The organic phase was isolated, dried (MgSO₄) and concentrated to give a dark solid 69 mg. MS (FAB⁺) 972 (M⁺), found 972.188507 (calculated for C₄₈H₅₂N₃OSI₂ 972.192065); WV-Vis (DCM) λ₍ₘₐₓ₎ 436, 522, 560, 697 nm

### 5-(4-hexadecyloxyphenyl)-10-(phenyl)-15,20-(2-ethynylpyridine)-21-thiaporphyrin [10]

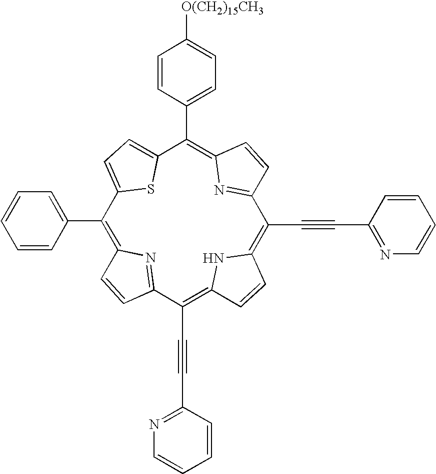

To a degassed solution of porphyrin (9) (70 mg, 72 μmol) in anhydrous toluene/triethylamine (5:1, 18 ml) tris(dibezylidineacetone)dipalladium(0) (10 mg, 11 μmol), followed by tris(o-toly)phosphine (26 mg, 86 μmol) was added. Then 2-ethynylpyridine (29 mg, 0.28 mmol) was added and the reaction mixture stirred at 40° C. under argon and shielded from light for 2 h. The reaction mixture was then concentrated and the residue purified by preparative TLC (silica gel: 70% THF/hexanes) to give the desired product 18 mg (27%). ¹H NMR δ (ppm, THF) 9.98 (m, 2H, thiophene-H); 9.65-9.53 (m, 2H, pyrrole-H), 9.45 (m, 2H, pyrrole-H), 8.83 (m, 2H, pyrrole-H), 8.65 (dd, 2H, J=10.1, J=5.3 Hz, pyridine-H), 8.23 m, 2H, phenyl-H), 8.17-8.09 (m, 3H, phenyl-H, pyridine-H), 7.94 (m, 2H, pyridine-H); 7.82 (m, 3H, phenyl-H), 7.48-7.36 (m, 5H, phenyl-H, pyridine-H), 4.23 (t, 2H, J=7 Hz, O—CH₂), 1.98 (m, 2H, —CH₂), 1.3-1.7 (m, 27H, —CH₂), 0.89 (t, 3H, J=7 Hz, —CH₃), −2.3 (s, 1H, NH); MS (FAB⁺) m/z 922 (M⁺, 100%) found 922.451614 (calculated for C₆₂H₆₀₀N₅OS 922.451859 (M+1);UV-Vis (THF) λ₍ₘₐₓ₎ 456, 537, 589, 658, 707, 724 nm

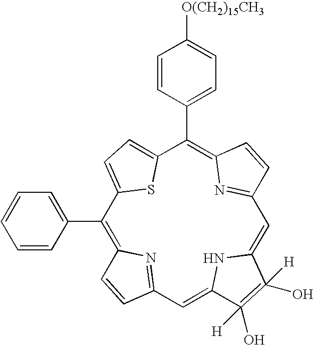

The core-modified porphyrin (8) (28 mg, 38.8 μmol) was dissolved in a mixture of chloroform and pyridine (10%, 1.5 ml) and osmium tetraoxide (12 mg, 46.5 μmol) was added. The mixture was stirred under nitrogen and in the dark for 2 h, during which time the reaction was monitored by UV/visible spectroscopy. The reaction was quenched by bubbling H₂S, concentrated in vacuo to give a dark green oil. This was purified by preparative TLC [silica gel: 20% EtOAc/DCM) and three products were isolated, two of these were identified as isomeric chlorins and the third as the isobacteriochlorin.

Fraction 1 (Chlorin): 8 mg (27%)

¹H NMR δ (ppm, CDCl₃) 9.72 (s, 1H), 9.59 (d, 1H, J=5 Hz), 9.4 (s, 1H), 9.26 (d, 1H, J=5 Hz), 9.15 (d, 1H, J=5 Hz), 9.03 (d, 1H, J=5 Hz), 8.84 (d, 1H, J=4 Hz), 8.60 (d, 1H, J=4 Hz), 8.19 (br s, 2H), 7.80 (m, 4H), 7.20 (m, 2H), 6.40 (m, 1H), 6.15 (m, 1H), 4.20 (m, 3H), 2.8 (m, 1H), 1.95 (m, 2H), 1.2-1.7 (m, 26H), 0.9 (m, 3H), −2.85 (br s, 1H); MS (FAB⁺) m/z 754.403809 calculated for C₄₈H₅₅N₃O₃S 754.404240; UV-Vis (DCM) λ₍ₘₐₓ₎ 413, 505, 531, 607, 661 nm

Fraction 2 (Chlorin): 6 mg (20%)

¹H NMR δ (ppm, CDCl₃) 9.60 (d, 1H, J=5 Hz), 9.44 (m, 1H), 9.30 (s, 1H), 9.20 (d, 1H, J=5 Hz), 8.94 (m, 2H), 8.66 (m, 1H), 8.50 (m, 1H), 8.04 (m, 3H), 7.70 (m, 4H), 7.30 (m, 2H), 6.35 (m, 1H), 6.05 (m, 1H), 4.45 (m, 1H), 4.20 (t, 2H, J=6 Hz), 3.0 (m, 1H), 2.0 (m, 2H), 1.2-1.7 (m, 26H), 0.93 (m, 3H), −3.4 (br s, 1H); (FAB⁺) m/z 754.403809 calculated for C₄₈H₅₅N₃O₃S 754.403689; UV-Vis (DCM) λ₍ₘₐₓ₎ 413, 505, 533, 607, 661 mm

Fraction 3 (Isobacteriochlorin) 2 mg (6%) (FAB⁺) m/z 787.40062 calculated for C₄₈H₅₇N₃O₅S 787.401894; UV-Vis (DCM) λ₍ₘₐₓ₎ 381, 518, 706 nm

2-Thiophene-2-Ylmethyl-1H-Pyrrole [15]

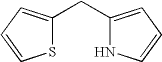

Ref: Bull. Kor. Chem. Soc., 1997, 18(2), 222

2-Thiophenemethanol (14) (3.30 g, 28.9 mmol) was stirred in pyrrole (38.78 g, 578 mmol) under Ar at 10° C. and BF₃.OEt₂ (6.14 g, 43.3 mmol, 5.3 ml) was added. The mixture was stirred for 45 mins then diluted with DCM (100 ml). The dark brown mixture was washed with aqueous NaOH (0.1M, 2×100 ml), then water (3×50 ml). The organic phase was then dried (MgSO₄) filtered and concentrated to give a dark brown oil. The product was purified by column chromatography with 20% ethyl acetate/hexane to give a pale brown oil (20%, Rf=0.45). ¹H NMR δ (ppm, CDCl₃) 7.92 (br s, 1H, NH), 7.17 (dd, 1H, J=5.2 and 1.2 Hz, thiophene-H), 6.94 (dd, 1H, J=4.9 and 3.2 Hz, thiophene-H), 6.85 (m, 1H, thiophene-H), 6.68 (dd, 1H, J=4.2 and 2.7 Hz, pyrrole-H), 6.16 (dd, 1H, J=5.9 and 2.9 Hz, pyrrole-H), 6.05 (m, 1H, pyrrole-H), 4.17 (s, 2H, CH₂); MS (EI⁺) m/z 163 (M⁺, 100%); found 163.045230 (calculated for C₉H₉NS 163.045571).

### 5-Thiopheit-2-ylmethyl-1H-pyrrole-2-carbaldehyde [16]

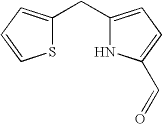

Vilsmeir reagent: Dimethyl formamide (10 ml) was stirred at 0° C. under Ar and POCl₃ (1.5 ml) was added in a drop wise manner. The solution was stirred for 20 min.

To a stirred solution of 2-thiophene-2-ylmethyl-1H-pyrrole (15) (0.80 g, 4.9 mmol) in anhydrous DMF (10 ml), at 0° C. and under Ar was added the Vilsmeir reagent (3 ml) in a drop wise manner. The resulting mixture was stirred for 3 h allowing the mixture to warm slowly to room temperature. The reaction mixture was added to aqueous NaOH (1M, 100 ml) and thoroughly mixed before extraction with ethyl acetate (3×5 ml). The combined organic extracts were washed with water (3×50 ml) then brine (50 ml). The organic phase was then dried (MgSO₄) filtered and concentrated to give a dark brown oil. Purification by column chromatography with 30% ethyl acetate/hexane gave 5-thiophen-2-ylmethyl-1H-pyrrole-2-carbaldehyde (16) as a pale brown solid (66%, Rf=0.4). ¹H NMR δ (ppm, CDCl₃) 9.95 (br s, 1H, NH), 9.40 (s, 1H, CHO), 7.19 (dd, 1H, J=5.1 and 1.2 Hz, thiophene-H), 6.95 (dd, 1H, J=5.1 and 3.4 Hz, thiophene-H), 6.92 (dd, 1H, J=3.8 and 2.5 Hz, pyrrole-H), 6.89 (m, 1H, thiophene-H), 6.17 (m, 1 H, pyrrole-H), 4.23 (s, 2H, CH₂); MS (EI⁺) m/z 191 (M⁺, 100%), 162 (80); found 191.040366 (calculated for C₁₀H₉NOS 191.040486).

### 5-[5-(4-Fluoro-benzoyl)-thiophen-2-ylmethyl]-1H-pyrrole-2-carhaldehyde [17]

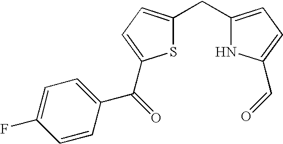

To a stirred slurry of AlCl₃ (558 mg, 4.18 mmol) in anhydrous dichloromethane (50 ml) at 0° C. and under Ar was added drop wise 4-fluorobenzoyl chloride (587 mg, 3.70 mmol, 0.44 ml). The mixture was stirred at 0° C. for 20 min. To this mixture was added a sample of 5-thiophen-2-ylmethyl-1H-pyrrole-2-carbaldehyde (16) (308 mg, 1.61 mmol) dissolved in anhydrous dichloromethane (5 ml). The mixture was then stirred for 5 h at room temperature. The crude reaction mixture was then filtered through a pad of silica, and the resulting solution pre-adsorbed onto silica. This was loaded on to a packed chromatography column and purified by elution with 40% ethyl acetate/hexane (85%, Rf=0.3). ¹H NMR δ (ppm, CDCl₃) 10.42 (br s, 1H, NH), 9.38 (s, 1H, CHO), 7.84 (m, 2H, Ar—H), 7.44 (m, 1H, thiophene-H), 7.14 (m, 2H, Ar—H), 6.93 (m, 2H, thiophene-H and pyrrole-H), 6.20 (dd, 1H, J=3.7 and 2.5 Hz, pyrrole-H), 4.30 (s, 2H, CH₂); MS (EI⁺) m/z 313 (M⁺, 100%), 284 (57), 190 (38), 123 (77); found 313.056977 (calculated for C₁₇H₁₂NO₂FS 313.057279).

### (4-Fluoro-phenyl)-[5-(5-hydroxylmethyl-1H-pyrrol-2-ylmethyl)-thiophen-2-yl]-metlhanol [19]

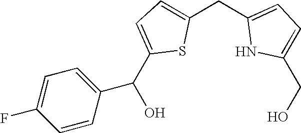

5-[5-(4-Fluoro-benzoyl)-thiophen-2-ylmethyl]-1H-pyrrole-2-carbaldehyde (17) (398 mg, 1.27 mmol) was dissolved in THF/methanol (1:1, 50 ml) and sodium borohydride (2.04 g, 53.9 mmol) was added in small portions over 30 mins. The reaction mixture was stirred at room temperature for a further 30 mins then poured into water (100 ml). The mixture was extracted with ethyl acetate (3×50 ml) and the combined organic extracts were washed with water (100 ml) then dried (K₂CO₃), filtered and concentrated to give a pale brown oil. As this product was unstable it was used immediately for the next step without purification.

### 5-(4-Fluorophenyl)-10,15,20-H-21-thiaporphyrin [21]

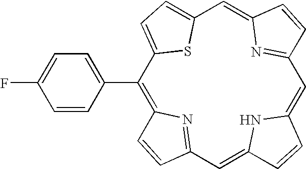

A mixture of (4-fluoro-phenyl)-[5-(5-hydroxyhnethyl-1H-pyrrol-2-ylnethyl)-thiophen-2-yl]-methanol (18) (401 mg, 1.27 mmol) and unsubstituted dipyrromethane (7) (185 mg, 1.27 mmol) were dissolved in acetonitrile (50 ml) and stirred under Ar. Ammonium chloride (679 mg, 12.7 mmol) and BF₃.OEt₂ (180 mg, 1.27 mmol, 0.156 ml) were added and the mixture was stirred for 30 mins in the dark. DDQ (576 mg, 2.54 mmol) and triethylamine (˜2 ml) were then added and the mixture was stirred in open air for a further 2 h. The reaction mixture was then concentrated, re-dissolved in dichloromethane and filtered through a pad of silica gel. The filtrate was concentrated and purified by flash column chromatography with 80% dichloromethane/hexane as eluent (5.6%, Rf=0.59). ¹H NMR δ (ppm, CDCl₃) 10.82 (s, 1H, meso-H), 10.34 (s, 1H, meso-H), 10.29 (s, 1H, meso-H), 10.13 (d, 1H, J=5 Hz, thiophene-H), 9.93 (d, 1H, J=5.1 Hz, thiophene-H), 9.59 (m, 2H, pyrrole-H), 9.25 (m, 3H, pyrrole-H), 8.87 (d, 1H, J=4.4 Hz, pyrrole-H), 8.24 (m, 2H, Ar—H), 7.55 (t, 2H, J=8.6 Hz, Ar—H), −3.61 (s, 1H, NH); MS (FAB⁺) m/z 421 (M⁺, 47%), 422 (M+1, 96%); found 421.104691 (calculated for C₂₆H₁₆N₃FS 421.104898) and 422.112801 (calculated for C₂₆H₁₇N₃FS 421.104898 (M+1)).

The following example was prepared using the method outlined above.

### 5-[5-(4-Methoxy-benzoyl)-thiophen-2-ylmetlylj-1H-pyrrole-2-carbaldehyde [18]

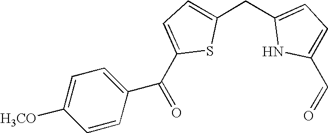

With p-anisoyl chloride and 5-thiophen-2-ylmethyl-1H-pyrrole-2-carbaldehyde (2), 5-[5-(4-Methoxy-benzoyl)-thiophen-2-ylmethyl]-1H-pyrrole-2-carbaldehyde (3a) was obtained. Purified by column chromatography with 30% ethyl acetate/hexane as eluent to give a green solid. (34%, Rf=0.51). ¹H NMR 5 (ppm, CDCl₃) 10.22 (br s, 1H, NH), 9.40 (s, 1H, CHO), 7.87 (d, 2H, J=8.8 Hz, Ar—H), 7.47 (d, 1H, J=3.7 Hz, thiophene-H), 6.96 (d, 2H, J=8.8 Hz, Ar—H), 6.93 (m, 2H, thiophene-H and pyrrole-H), 6.20 (m, 1H, pyrrole-H), 4.30 (s, 2H, CH₂), 3.88 (s, 3H, OCH₃); MS (FAB⁺) m/z 326 (M⁺, 100%); found 325.077667 (calculated for C₁₈H₁₅NO₃S 325.077265).

### [5-(5-Hydroxymethyl-H-pyrrol-2-ylmethyl)-thiophene-2-yl]-4(-methoxy-phenyl)-methanol [20]

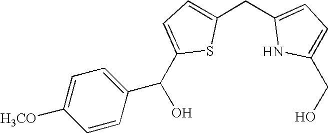

Reaction of 5-[5-(4-Methoxy-benzoyl)-thiophen-2-ylmethyl]-1H-pyrrole-2-carbaldehyde (18) gave [5-(5-Hydroxymethyl-lH-pyrrol-2-ylmethyl)-thiophene-2-yl]-4(-methoxy-phenyl)-methanol (20) as a yellow oil. This was used without purification.

### 5-(4-Methoxyphenyl)-10,15,20-H-21-tliiaporphyrin [22]

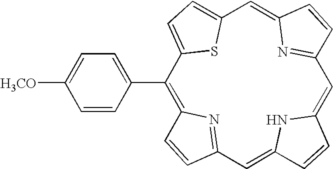

Reaction of [5-(5-Hydroxymethyl-1H-pyrrol-2-ylmethyl)-thiophene-2-yl]-4(-methoxy-phenyl)-methanol ((20) and unsubstituted dipyrromethane (7) gave porphyrin (5a). Purified by flash column chromatography with 80% dichloromethane/hexane as eleunt. (4.3%, Rf=0.47). ¹H NMR δ (ppm, CDCl₃) 10.80 (s, 1H, meso-H), 10.34 (s, 1H, meso-H), 10.27 (s, 1H, meso-H), 10.13 (d, 1H, J=5.3 Hz, thiophene-H), 9.99 (d, 1H, J=5.0 Hz thiophene-H), 9.59 (m, 2H, pyrrole-H), 9.23 (m, 3H, pyrrole-H), 8.93 (d, 1H, J=4.4 Hz, pyrrole-H), 8.22 (d, 2H, J=8.6 Hz, Ar—H), 7.39 (d, 2H, J=8.6 Hz, Ar-H), 4.12 (s, 3H, OCH₃), −3.57 (s, 1H, NH); MS (FAB⁺) n/z 434 (M+1, 20%); found 434.133286 (calculated for C₂₇H₂₀N₃SO 434.132709 (M+1)).

### Phenyl-(5-thiophene-2-ylmethyl-1H-pyrrole-2-yl)-methanone [28]

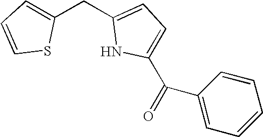

2-Thiophene-2-ylmethyl-lH-pyrrole (15) (1.011 g, 6.19 mmol) was dissolved in anhydrous THF (50 ml) under Ar at 0° C. Ethyl magnesium bromide (1M solution in THF) (30.9 ml, 30.9 mmol) was added drop wise and the resulting mixture was stirred for 30 min at 0° C. Benzoyl chloride (1.74 g, 12.4 mmol, 1.43 ml) was then added drop wise keeping the temperature below 5° C. After addition the mixture was allowed to warm to room temperature and was stirred for a further 2 h before carefully being quenched with satd. NH₄Cl solution (100 ml). The mixture was extracted with ethyl acetate (2×50 ml) and the combined organic extracts washed with water (2×50 ml) then brine (50 ml). The organic phase was then separated and dried (MgSO₄) filtered and concentrated to give a dark yellow oil. The crude product was purified by column chromatography with 20% ethyl acetate/hexane as eluent to give the desired monoacylated pyrromethane as a yellow solid. Further purification by recrystallisation from hexane gave a white solid. (48%, Rf=0.28). ¹H NMR δ (ppm, CDCl₃) 9.81 (br s, 1H, NH), 7.85 (m, 2H, Ar—H), 7.53-7.42 (m, 3H, Ar—H), 7.17 (dd, 1H, J=4.9 Hz and J=1.2 Hz, thiophene-H), 6.94 (dd, 1H, J=5.2 Hz and J=3.4 Hz, pyrrole-H), 6.89 (dd, 1H, J=3.5 Hz and J=1.0 Hz, thiophene-H), 6.80 (dd, 1H, J=3.7 Hz and J=1.2 Hz, thiophene-H) 6.15 (m, 1H, pyrrole-H), 4.26 (s, 2H, CH₂); MS (EI⁺) m/z 267 (M⁺, 100%), 190 (8), 162 (93), 105 (18), 77 (26); found 267.072229 (calculated for C₁₆H₁₃NSO 267.071786).

### [5-(5-Benzoyl-1H-pyrrol-2-ylmethyl)-thiophen-2-yl]-phenyl-methanone [29]

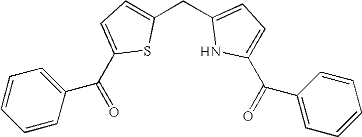

To a stirred slurry of AlCl₃ (694 mg, 5.21 mmol) in anhydrous dichloromethane (50 ml) at 0° C. and under Ar was added drop wise benzoyl chloride (732 mg, 5.21 mmol). The mixture was stirred at 0° C. for 20 min. To this mixture was added a sample of phenyl-(5-thiophene-2-ylmethyl-1H-pyrrole-2-yl)-methanone (28) (774 mg, 2.90 mmol) dissolved in anhydrous dichloromethane (5 ml). The mixture was then stirred for 16 h at room temperature then pre-adsorbed onto silica. The resulting solid was purified by flash column chromatography with 30% ethyl acetate/ hexane as eluent (37%, Rf=0.46). ¹H NMR δ (ppm, CDCl₃) 10.26 (br s, 1H, NH), 7.90-7.77 (m, 4H, Ar—H), 7.60-7.50 (m, 2H, Ar—H), 7.50-7.42 (m, 5H, thiophene-H and Ar—H), 6.92 (d, 1H, J=3.7 Hz, pyrrole-H), 6.82 (dd, 1H, J=3.7 Hz and J=2.5 Hz, thiophene-H), 6.20 (m, 1H, pyrrole-H), 4.33 (s, 2H, CH₂); MS (EI⁺) m/z 371 (M⁺, 100%), 266 (74), 105 (43), 77 (27); found 371.098520 (calculated for C₂₃H₁₇NO₂S 371.098001).

### {5-[5-(Hydroxy-phenyl-nietlyl)-1H-pyrrole-2-ylmethyl]-thiophen-2-yl}-phenyl-methanol [30]

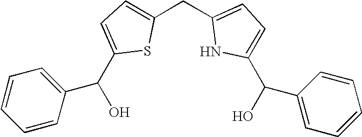

[5-(5-Benzoyl-lH-pyrrol-2-ylmethyl)-thiophen-2-yl]-phenyl-methanone (29) (370 mg, 0.99 mmol) was dissolved in THF/methanol (1:1, 50 ml) and sodium borohydride (1.88 g, 49.8 mmol) was added in small portions over a 20 min period. The reaction mixture was stirred at room temperature for a further 1h then poured into water (100 ml). The mixture was extracted with ethyl acetate (2×50 ml) and the combined organic extracts were washed with water (100 ml) then dried (K₂CO₃), filtered and concentrated to give a yellow oil. As this product was unstable it was used immediately for the next step without purification.

### 5,15-Diphenyl-10,20-H-21-thiaporphyrin [31]

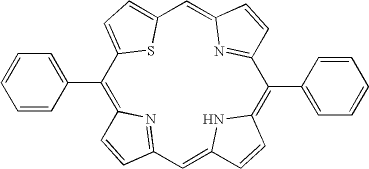

A mixture of {5-[5-(Hydroxy-phenyl-methyl)-1H-pyrrole-2-ylmethyl]-thiophen-2-yl}-phenyl-methanol (30) (370 mg, 0.99 mmol) and unsubstituted dipyrromethane (7) (144 mg, 0.99 mmol) were dissolved in acetonitrile (50 ml) and stirred under Ar. Ammonium chloride (529 mg, 9.9 mmol) and BF₃.OEt₂ (140 mg, 0.99 mmol, 0.121 ml) were then added and the mixture was stirred for 30 mins in the dark. DDQ (449 mg, 1.98 mmol) and triethylamine (˜2 ml) were then added and the mixture was stirred in the open air for 2 h. The reaction mixture was then concentrated, re-dissolved in dichloromethane and filtered through a silica gel plug. The filtrate was concentrated and purified by flash column chromatography with 80% dichloromethane/hexane as eluent (7.5%, Rf=0.51). ¹H NMR δ (ppm, CDCl₃) 10.79 (s, 1H, meso-H), 10.32 (s, 1H, mneso-H), 10.09 (d, 1H, J=5.1 Hz, thiophene-H), 9.95 (d, 1H, J=5.1 Hz, thiophene-H), 9.48 (dd, 1H, J=4.8 Hz and J=1.8 Hz, pyrrole-H), 9.20 (d, 1H, J=4.5 Hz, pyrrole-H), 9.15 (dd, 1H, J=4.8 Hz and J=1.8 Hz, pyrrole-H), 9.12 (d, 1H, J=4.5 Hz, pyrrole-H), 8.88 (d, 1H, J=4.2 Hz, pyrrole-H), 8.78 (d, 1H, J=4.5 Hz, pyrrole-H), 8.40-8.22 (m, 4H, Ar—H), 7.90-7.78 (m, 6H Ar—H), −3.14 (s, 1H, NH); MS m/z (FAB⁺) 479 (M⁺), found 479.146828 (calculated for C₃₂H₂₁N₃S 479.145620) and 480.153442 (calculated for C₃₂H₂₂N₃S, (M+1); UV-Vis (DCM) λ₍ₘₐₓ₎ 417, 502, 532, 601, 661 nm

### 5-(4-Hydroxyphenyl)-10,15,20-H-21-thiaporphypin [36]

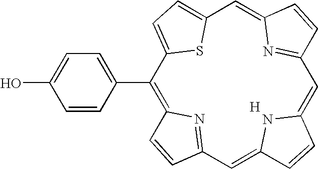

A solution of the 4-methoxyphenylthiaporphyrin (22) in dry DCM (10 ml) was cooled to 0° C. under argon and borontribromide (lM solution in DCM, 353 μl ) was added and the emerald green solution. stirred at room temperature. A TLC [silica gel: 10% EtOAc/chloroform) after 5 h showed a new product spot (lower R_{f} 0.51) a spot corresponding to the starter (R_{f} 0.84). The reaction mixture was cooled back down to 0° C. and a further 706 μl of BBr₃ was added and the reaction stirred at room temperature for a further 12 h after which it was quenched by pouring into a mixture of ethylacetate (100 ml) and satd. NaHCO₃ solution (50 ml). A further 100 ml of ethyl acetate was added to the reaction mixture to aid dissolution, the organic layer separated, dried over MgSO₄ and vac down to give a purple solid 29 mg (100%); MS (FAB⁺) m/z 420 (M+1, 10%); UV-Vis (EtOAc) λ₍ₘₐₓ₎ 410, 495, 523, 597, 656 nm.

### 5-Ethyl(4-carboxylatometlioxyphenyl)-10,15,20-H-21-thiaporphyrin [37]

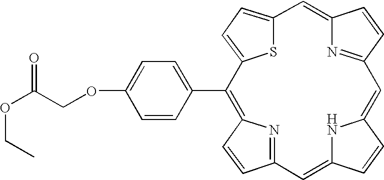

The 4-Hydroxyphenylthiaporphyrin (36) (29 mg, 0.069 mmol) was dissolved in anhydrous DMF (10 ml) and placed under an argon blanket. To this solution anhydrous potassium carbonate (0.5 g, 3.46 mmol) was added followed by ethylbromoacetate (0.29 g, 1.73 mmol). The resulting mixture was stirred at 50° C. for 12 h, cooled to room temperature and poured into a large volume of ice/water. This was extracted with DCM (2×100 ml), the combined extracts washed well with water, dried over MgSO₄ and evaporated to give a purple solid 29 mg (83%). TLC [silica gel: chloroform, R_{f} 0.24]; ¹H NMR δ (ppm, CDCl₃) 10.80 (s, 1H, meso-H), 10.34 (s, 1H, meso-H), 10.27 (s, 1H, meso-H), 10.13 (d, 1H, J=5.3 Hz, thiophene-H), 9.99 (d, 1H, J=5.0 Hz thiophene-H), 9.59 (m, 2H, pyrrole-H), 9.23 (m, 3H, pyrrole-H), 8.93 (d, 1H, J=4.4 Hz, pyrrole-H), 8.22 (d, 2H, J=8.6 Hz, Ar—H), 7.39 (d, 2H, J=8.6 Hz, Ar—H), 4.92 (s, 2H, CH₂), 4.41 (q, 2H, J=7 Hz), 1.4 (t, 3H, J=7 Hz); MS (FAB⁺) m/z 506 (M+1, 25%); UV-Vis (DCM) λ₍ₘₐₓ₎ 414, 497, 525, 596, 656 nm.

### 5-Ethyl(4-carboxylatomethoxyphenyl)-10,15, 20-triiodo-21-thiaporphyrin [38]

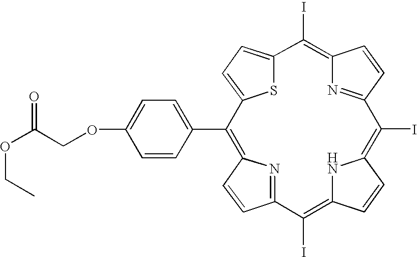

To a light protected solution of (CF₃CO₂)₂PhI (72 mg, 0.17 mmol) in dry CHCl₃ (10 ml) a solution of iodine (42 mg, 0.1 mmol) in dry chloroform (5 ml) was added followed by a few drops of pyridine. The resulting rnixture was stirred at room temperature under nitrogen until a pale yellow solution formed. This solution was then added drop wise to a light protected solution of the thiaporphyrin (37) (21 mg, 0.042 mmol) in anhydrous chloroform (25 ml). The addition took 10 min. after which the mixture was stirred for a further 24 h under nitrogen. A TLC [silica gel: 60% DCM/hexane showed the presence of mainly the diiodinated porphyrin with a small amount of the tri-substituted, this was confirmed by UV. A further 2 molar equivalents of the iodinating reagent was prepared and added to the reaction mixture which was then heated at approx 40-45° C. for 12 h. This helped push the triiodination as shown by TLC and UV. The reaction mixture was then diluted with chloroform (100 ml) and washed with saturated sodium thiosulphate solution (2×100 ml). The organic phase was isolated, dried (MgSO₄) and concentrated to give a dark solid 35 mg. MS (FAB⁺) 883 (M⁺).

### 5-Ethyl(4-carboxylatomethoxyphenyl)-10,15,20-(m-O-tbutyldimethylsilaile-ethynylphenol)-21-thiaporphyrin [39]

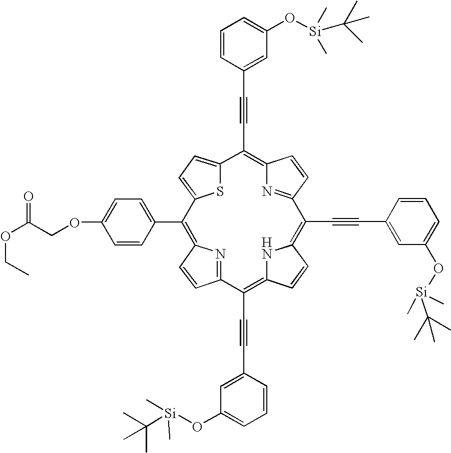

To a degassed solution of thiaporphyrin (38) (35 mg, 0.4 mmol) in anhydrous toluene/triethylamine (5:1, 18 ml) tris(dibezylidineacetone)dipalladium(0) (6 mg, 6 μmol), followed by tris(o-toly)phosphine (29 mg, 48 μmol) was added. Then 3-O-tbutyldimethylsilyl-ethynylphenol (83 mg, 0.4 mmol) was added and the reaction mixture stirred at 40° C. under argon and shielded from light for 6 h. The reaction mixture was then concentrated and the residue purified by preparative TLC (silica gel: 70% THF/hexanes) to give the desired product 12 mg (26%); MS (FAB⁺) m/z 1198 (M⁺, 20%); UV-Vis (THF) λ₍ₘₐₓ₎ 460, 575, 707, 749 nm

### 5-Ethyl(4-carboxylatomethoxyphenyl)-10,15,20-(m-ethynylphenol)-21-thiaporphyrin [40]

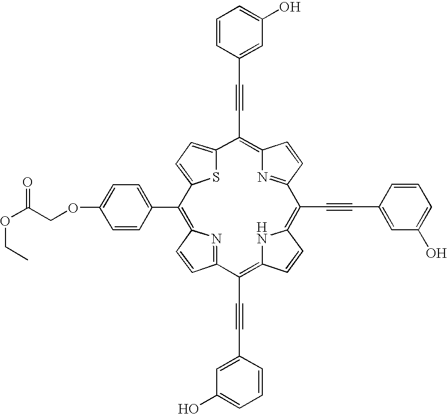

To a light-protected solution of thiaporphyrin (39) (12 mg, 10 μmol) in dry THF (1 ml) tetrabutylammonium fluoride (100 μl, 1M solution in THF) was added drop-wise and the resulting solution stirred at room temperature for 1 h. The reaction was monitored by TLC and on completion was evaporated to give a sticky solid which was washed with water and pumped down to dryness to give a green-brown solid (90%). MS (FAB⁺) m/z 856 (M⁺).

### 5-[5-(4-Iodo-benzoyl)-thiophen-2-ylmetlyl]-1H-pyrrole-2-carhaldelyde [44]

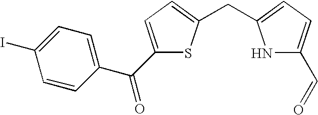

To a stirred slurry of AlCl₃ (0.72 g, 5.4 mmol) in anhydrous dichloromethane (50 ml) at 0° C. and under Ar was added drop wise 4-iodobenzoyl chloride (1.27 g, 4.8 mmol). To this cooled mixture was added drop-wise a sample of 5-thiophen-2-ylmethyl-1H-pyrrole-2-carbaldehyde (16) (0.4 g, 2.1 mmol) dissolved in anhydrous dichloromethane (5 ml). The mixture was then stirred overnight at room temperature. The light-brown crude reaction mixture was then evaporated to dryness and the resulting dark-blue sticky solid purified by column chromatography eluting with 30% ethyl acetate/ hexane (60%, Rf=0.36). ¹H NMR δ (ppm, CDCl₃) 9.82 (br s, 1H, NH), 9.40 (s, 1H, CHO), 7.85 (d, 2H, Ar—H, J=8.9 Hz), 7.54 (d, 2H, Ar—H, J=8.9 Hz), 7.44 (d, IH, J=3.7 Hz, thiophene-H), 6.93 (m, 2H, thiophene-H and pyrrole-H), 6.20 (dd, 1H, J=3.7 and 2.5 Hz, pyrrole-H), 4.28 (s, 2H, CH₂); MS (EI⁺) m/z 421 (M⁺, 100%).

### (4-Iodo-phenyl)-[5-(5-hydroxylmethyl-1H-pyrrol-2-ylmethyl)-thiophen-2-yl]-methanol [45]

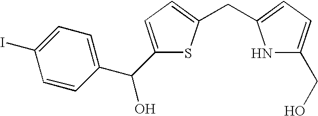

5-[5-(4-Iodobenzoyl)-thiophen-2-ylmethyl]-1H-pyrrole-2-carbaldehyde (44) (0.5 g, 1.18 mmol) was dissolved in THF/methanol (3:1, 60 ml) and sodium borohydride (2.3 g, 59 mmol) was added in small portions over 30 mins. The reaction mixture was then stirred at room temperature for a firter 30 mins, and poured into water (100 ml). The mixture was extracted with ethyl acetate (3×50 ml) and the combined organic extracts were washed with water (100 ml) then dried (K₂CO₃), filtered and concentrated to give a yellow-brown oil. The reduction was followed by TLC [neutral aluminium oxide: 5% MeOH/DCM, R_{f} 0.27 for the diol compared to R_{f} 0.78 for compound (41). As this product was unstable it was used immediately for the next step without purification.

### 5-(4-Iodophenyl)-10,15,20-H-21-thiaporphyrin [46]

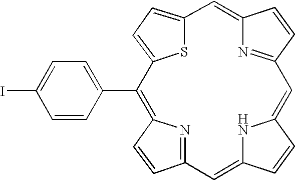

A mixture of (4-Iodophenyl)-[5-(5-hydroxylmethyl-1H-pyrrol-2-ylmethyl)-thiophen-2-yl]-methanol (45) (0.5 g, 1.19 mmol) and unsubstituted dipyrromethane (7) (0.17 g, 1.19 mmol) were dissolved in acetonitrile (70 ml) and stirred under a stream of Ar at 0° C. for approx. 10 min. Ammonium chloride (0.63 g, 11.8 mmol) followed by BF₃.OEt₂ (0.146 ml, 1.19 mmol) was added and the mixture stirred for 30 mins in the dark. DDQ (0.54 g, 2.34 mmol) and triethylamine (˜2 ml) were then added and the mixture was stirred in open air for a further 1 h. The dark green-brown reaction mixture wyas filtered through a pad of silica gel, eluting with DCM. The filtrate was concentrated and purified by flash column chromatography with 90% dichlorometaane/hexane as eluent (11%, Rf =0.70). ¹H NMR δ (ppm, CDCl₃) 10.82 (s, 1H, meso-H), 10.34 (s, 1H, meso-H), 10.29 (s, 1H, meso-H), 10.13 (d, 1H, J=5 Hz, thiophene-H), 9.93 (d, 1H, J=5.1 Hz, thiophene-H), 9.59 (m, 2H, pyrrole-H), 9.25 (m, 3H, pyrrole-H), 8.9 (d, 1H, 3=4.4 Hz, pyrrole-H), 8.21 (d, 2H, J=8.6, Ar—H), 7.42 (d, 2H, J=8.6 Hz, Ar—H), −3.61 (s, 1H, NH); MS (FAB⁺) m/z 529 (M⁺, 37%); UV-Vis DCM) λ₍ₘₐₓ₎ 411, 496, 525, 594, 655 nm

### 5-(4-Iodophetzyl)-10,15,20-tribromo-21-thiaporphyrin [47]

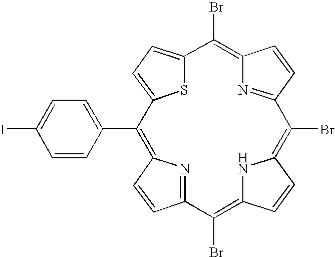

The thiaporphyrin (46) (20 mg, 37.8 μl) was dissolved in a mixture of dry chloroform and dry pyridine (30:1, 20 ml) and stirred under argon at 0° C. NBS (recrystallised from hot water, 20 mg, 113.4 μmol) was added and the mixture stirred at 0° C. for 2 h. The reaction mixture was then concentrated and purified by column chromatography [silica gel: 20% Hexane/DCM)] to give the tribrominated thiapoiphyrin 11 mg (55%). MS (FAB⁺) m/z 766 (M⁺, 40%).

### 5-[5-(4-Pentafluorobenzoyl)-thiophen-2-ylmethyl]-1H-pyrrole-2-carbaldehyde [50]

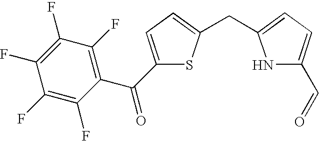

To a stirred slurry of AlCl₃ (0.56 g, 4.18 mmol) in anhydrous dichloromethane (40 ml) at 0° C. and under Ar was added drop wise 4-pentafluorobenzoyl chloride (0.53 ml, 3.7 mmol). To this cooled mixture was added drop-wise a sample of 5-thiophen-2-ylmethyl-1H-pyrrole-2-carbaldehyde (16) (0.31 g, 1.62 mmol) dissolved in anhydrous dichloromethane (5 ml). The mixture was then stirred overnight at room temperature, during this time a sticky substance came out of solution. The light-brown crude reaction mixture was then evaporated to dryness and the resulting light-brown sticky solid purified by column chromatography eluting with 30% ethyl acetate/ hexane (50%, Rf=0.34). ¹H NMR δ (ppm, CDCl₃) 9.88 (br s, 1H, NH), 9.50 (s, 1H, CHO), 7.54 (m, 1H,, thiophene-H), 6.93 (m, 2H, thiophene-H and pyrrole-H), 6.20 (m, 1H, pyrrole-H), 4.29 (s, 2H, CH₂); MS (EI⁺) m/z 385 (M⁺, 100%).

### 5-(5-Hydroxynethyl-1H-pyrrol-2-ylmethyl)-tliophene-2-yl]-4(-methoxy-phenyl)-methanol [51]

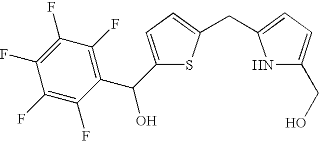

5-[5-(Pentafluorobenzoyl)-thiophen-2-ylmethyl]- 1H-pyrrole-2-carbaldehyde (50) (0.3 g, 0.8 mnmol) was dissolved in THE/methanol (3:1, 40 ml) and sodium borohydride (1.47 g, 39 mmol) was added in small portions over 20 mins. The reaction mixture was then stirred at room temperature for a further 30 mins, and poured into water (100 ml). The mixture was extracted with ethyl acetate (3×50 ml) and the combined organic extracts were washed with water (100 ml) then dried (K₂CO₃), filtered and concentrated to give a yellow-brown oil. The reduction was followed by TLC [neutral alurinium oxide: 5% MEOH/DCM. As this product was unstable it was used immediately for the next step without purification.

### 5-(Pentafluorophenyl)-10,15,20-H-21-thiaporphyrin [52]

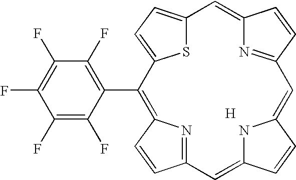

A mixture of 5-Pentafluorophenyl)-[5-(5-hydroxylmethyl-1H-pyrrol-2-ylmethyl)-thiophen-2-yl]-methanol (51) (0.3 g, 0.8 mmol) and unsubstituted dipyrromethane (7) (0.117 g, 0.8 mmol) were dissolved in acetonitrile (50 ml) and stirred under a stream of Ar at 0° C. for approx. 10 min. Ammonium chloride (0.63 g, 8.0 mmol) followed by BF₃.OEt₂ (98 μl, 0.8 mmol) was added and the mixture stirred for 30 mins in the dark. DDQ (0.36 g, 1.60 mmol) and triethylamine (˜2 ml) were then added and the mixture was stirred in open air for a further 1 h. The dark green-brown reaction mixture was filtered through a pad of silica gel, eluting with DCM. The filtrate was concentrated and purified by flash column chromatography with 90% dichloromethane/hexane as eluent (5%). MS (FAB⁺) m/z 493 (M⁺, 15%); UV-Vis (DCM) λ₍ₘₐₓ₎ 410, 494, 526, 594, 655 nm

Evaluation of in vitro Photodvnamic Tumour Cell Kill

Preparation of Tumour Cells:

The non-metastasing mouse colon adenocarcinoma cell line Colo26 was maintained in monolayer culture in RPMI 1640 glutamax medium supplemented with 10% heat-inactivated foetal calf serum, 100 U ml⁻¹ penicillin and 100 mg ml⁻¹ streptomycin. Cells grown to confluence were separated by trypsinisation to produce a suspension of 1.39 million cells per ml. A sample of 2.16 ml of this cell suspension was diluted to 50 ml with CM10 medium to give suspensions of 60,000 cells per ml. 100 μl of this suspension containing 6000 cells was added to each of the 60 inner wells of two 96-well culture plates. The outer wells of each plate were filled with sterile water. This was done to avoid errors resulting from the loss by evaporation of the rather small volumes of cell culture medium. The cells were incubated at 37° C. and 5% CO₂ in a humidified incubator for 24 hours. A further 100 μl of cell culture medium containing a range of concentrations of each photosensitiser and tetrahydoflran (THF) vehicle was then added to groups of six wells. Tetrahydrofuran was chosen as solvent because it is relatively freely miscible with water and a pilot experiment had shown that incubation of cells for 48 hours with 0.5-1% THF had no effect on cell growth. An initial stock solution of 200 μl of each photosensitiser in THF was prepared and this solution used to prepare a series of sub-stock solutions in cell culture medium CM10 for addition to the tumour cell cultures. The final concentration of each photosensitiser and THF vehicle incubated with each group of six wells containing the tumour cells are shown Table 1.

TABLE 1
Concentration of photosensitiser and vehicle incubated with cells

| Compound | Concentration after addition to cell culture | | | | | | | | |
|---|---|---|---|---|---|---|---|---|---|
| Photosensitiser (μM) | 2 | 1 | 0.5 | 0.1 | 0.05 | 0.01 | 0 | 0 | 0 |
| THF % (v:v) | 1.0 | 0.5 | 0.25 | 0.05 | 0.025 | 0.005 | 1.0 | 0.5 | 0 |

Photoactivation

After 20 hours of incubation with the cells, the medium containing the sensitiser and vehicle was removed and the cells washed once with buffer solution at 37° C. before the addition of fresh cell culture medium without photosensitiser or vrhicle. One of the two cell culture plates was then protected from light with aluminium foil and placed alongside the other plate which was irradiated with 100 joules cm⁻² of full-spectrum white light from an Applied Photophysics Irradiator. The light was delivered to the base of the 96-well plate through a glass platform (which served to filter short-wavelength ultraviolet light) as a 20 cm diameter spot. After irradiation both plates were returned to the incubator for a further 18 hours.

Cell growth inhibition following incubation with each photosensitiser and irradiation was determined using the European Union approved Neutral Red photocyiotoxicity protocol (Annexe II to Directive 2000/33/EC) adapted for visible light irradiation and the use of colo26 cells.

Phototoxicitv Results

The mean cell survival of each group of cells incubated with porphyrin photosensitiser (8), (10), and (11) in the dark or with irradiation with 100 J cm^{2 -}white light are shown in FIGS. 1-3.

Conclusions

These studies have shown that porphyrin sensitisers (8), (10) and (11) have photodynamic activity. There is no cytotoxcitity after 20 hours of incubation of colo26 cells with up to 2 μM porphyrins (8) and (10) in the absence of light, but substantial cell kill by concentrations of 0.5 μM and above on irradiation with 100 J cm⁻² white light, approaching total cell kill at 2 μM and above. Porphyrin (11) also shows no cytoxicitity in the absence of light after 20 hours incubation with colo26 tumour cells ay concentrations up to 10 μM. On irradiation with 100 J cm⁻² white light, porphyrin (11) caused almost total cell kill at concentrations of 5 μM and above.

Spectroscopic Properties

FIG. 4 shows the molar extinction spectrum of porphyrin (8) in THF. The absorbance peaks and molar extinction coefficients are shown below in Table 2.

TABLE 2

| Peak Wavelength | ε (1 mol⁻¹ cm⁻¹) |
|---|---|
| 418 | 300,000 |
| 502 | 29,800 |
| 532 | 5,300 |
| 606 | 4,900 |
| 668 | 3,000 |

FIG. 5 shows the molar extinction spectrum of porphyrin (10) in THF. The Absorbance peaks and molar extinction coefficients are shown below in Table 3.

TABLE 3

| Peak Wavelength | ε (1 mol⁻¹ cm⁻¹) |
|---|---|
| 456 | 378,985 |
| 537 | 24,330 |
| 589 | 40,385 |
| 659 | 6,335 |
| 708 | 11,635 |
| 724 | 13,320 |

FIG. 6 shows the molar extinction spectra of porphyrin (11) and (12) in THF [solid line porphyrin (11) and dashed line porphyrin (12)]. The absorbance peaks and molar extinction coefficients are shown below in Table 4.

TABLE 4

ε (1 mol⁻¹ cm⁻¹)

| Peak Wavelength | Porphyrin (11) | Porphyrin (12) |
|---|---|---|
| 413 | 95,472 | 106,175 |
| 505 | 12,090 | 12,796 |
| 531 | 5,593 | 6,756 |
| 607 | 2,904 | 2,984 |
| 661 | 13,558 | 14,085 |

Determination of Singlet Oxygen Quantum Yields (Φ)

Air equilibrated solutions of the sensitisers were optically matched at the laser excitation wavelength along with that of the reference standard whose singlet oxygen quantum yield is known. Singlet oxygen generation is detected by its phosphorescence at 1270 nm following laser excitation. At each laser intensity the recorded phosphorescence trace was obtained by signal averaging 10 single shots. A linear regression between the signal amplitude and the laser intensity is carried out with the aim of calculating the slope of the straight line and since the gradient is proportional to the singlet oxygen quantum yield, by comparison with the gradient obtained for the standard, the singlet oxygen quantum yield for the sample can calculated using:
Φ=Φ_{standard}×(slopeₛₐₘₚᵢₑ/slope_{standard})×(absorptionₛₐₘₚₗₑ/absorption_{standard})

This is a comparative method where the value obtained for Φ is relative to that of a known standard. The quality of the results obtained are heavily dependent on the quality of the standard, which should be freshly prepared before measurements.

The singlet oxygen quantum yield for porphyrin (11) in toluene was 0.985822

The singlet oxygen quantum yield for reference (chlorophyll A) in toluene was 0.6

Svnthesis and Utility of scFv-4 Helix Bundle Fusion Protein Carrying PS Drug Molecules

A chosen, well characterised scFv is PCR amplified and cloned as an Nco I/Not I fragment into the bacterial expression vector pET20b (Novagen) to create pETscFv. A DNA cassette containing a 4 helix bundle (e.g. a derivative of the bacterial protein ‘rop’) is PCR amplified and cloned into the Not I site of pETscFv to create pETscFv4HB (FIG. 9). Appropriate DNA primers are used introduce cysteine residues at optimal positions in the helix bundle and to replace any cysteine residues in the scFv (with residues which do not significantly alter the binding characteristics of the scFv, such as serine, alanine and glycine). The resulting construct is called pETscFv4HBcys

The vector pETscFv4HBcys is transformed into E. coli BL21 (DE3) (Novagen) by the calcium chloride method and plated onto 2TY agar plates containing 100 μg/ml ampicillin [Sambrook et al. (1989). DNA Cloning. A Laboratory Manual. Cold Spring Harbor]. Single colony transformants are picked and re-streaked onto fresh 2TY Agar plates containing amplicillin.

A single colony is picked and grown in 5 ml of 2TY media containing 100 μg/ml ampicillin at 30° C., in a shaking incubator (250 rpm) for 8-16 hr. This culture is then used to inoculate a culture of 500 ml 2TY media containing 100 μg/ml ampicillin and grown under similar conditions for a further 3-16 hr.

The culture supernatant is harvested and concentrated using an Amicon ultrafiltration stirred cell with a 30 KDa cut-off membrane to a final volume of 10 ml. Alternatively, the bacterial periplasm can be prepared using the sucrose osmotic shock method [Deonarain M P & Epenetos A A (1998) Br. J. Cancer. 77, 537-46. Design, characterization and anti-tumour cytotoxicity of a panel of recombinant, mammalian ribonuclease-based immnunotoxins] in a volume of 10 ml.

The concentrated supernatant or periplasmic extract is dialysed for 16 hr against 5 L of phosphate-buffered saline (PBS) containing 0.5 M NaCl and 2 mM MgCl₂. This is then applied to a copper (II) or nickel (II)-charged chelating sepharose column (Amersham-Phannacia Biotech) and purified by immobilised metal affinity chromatography (IMAC) for example as described in Deonarain et al [Deonarain M P & Epenetos A A (1998) Br. J. Cancer. 77, 537-46. Design, characterization and anti-tumour cytotoxicity of a panel of recombinant, mammalian ribonuclease-based immunotoxins]. The recombinant fusion protein should elute in an imidazole gradient at between 40 and 150 mM imidazole. The eluted fusion protein is further purified by gel filtration on a superdex-200 column (Amersham-Pharmacia Biotech) equilibrated in PBS. FIG. 10 shows shows data for the expression and purification of the resulting fusion protein, scFv-4-helix bundle-cys.

Preiaration of pETscFv4HBLvs

A scFv-4 helix bundle was prepared in accordance with the methodology described above, except that appropriate primers were used to introduce lysine residues at optimal positions in the helix bundle. The resulting construct is called pETscFv4HBLys. An scFv which targets CEA (carcinoembryonic antigen) was used.

Coupling of Photosensitiser to scFv4Helix Bundle-Lys

The N-hydroxysuccinimide (NHS) ester of a carboxyl-containing photosensitiser was prepared by reacting 1.5 equivalents of dicyclohexylcarbodiimide and 1.5 equivalents of NHS with one equivalent of photosensitiser in dry dimethyl sulphoxide (DMSO). The reaction was carried out under an inert gas (eg argon) and in the dark at room temperature and was complete in 2 hours, (tic: silica gel 3% methanol in chloroform). A similar procedure can be used to prepare the active ester of any carboxyl containing photosensitiser.

N-ethylmorpholine (1 μl), DMSO (10 ml) and the scFv4-helix bundle (100 μg in approx. lml of PBS buffer) were stirred together in the dark and under nitrogen at room temperature. To this solution was added the DMSO solution containing the photosensitiser-NHS ester. The solution was stirred at room temperature in the dark for 12 hours to synthesise the bundle photosensitiser conjugate. The conjugate was then dialysed against 2×5 SL of PBS. All procedures were carried out in the dark. Preferably, the coupling reaction between the photosensitiser and the scFv4-helix bundle is carried out at low temperatures in 5-10% DMSO diluted with buffer.

The number photosensitisers attached to the 4-helix bundle fusion protein is determined using electrospray mass spectrometry, compared to the 4-helix bundle alone. To confirm the position of attachment on the 4-helix bundle, the protein will be fragmented by trypsin digestion and the resulting peptides analysed by mass spectrometry.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in the relevant art are intended to fall within the scope of the following claims.

1. A compound of formula I, or a pharmaceutically acceptable salt thereof, 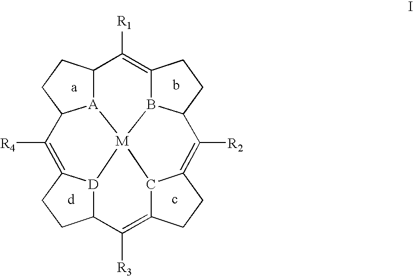
wherein
one or two of A, B, C and D are each independently selected from S, O, Se and Te, and the remainder are N;
a, b, c and d are each independently substituted or unsubstituted 5-membered heterocyclic groups having the members necessary to complete a porphyrin, chlorin, bacteriochlorin or isobacteriochlorin nucleus in which one or two of the nitrogens are replaced by S, O, Se or Te;
M is H or a metal;
R₁, R₂, R₃ and R₄ are each independently selected from:
H;
alkyl;
cycloalkyl;
halogen;
aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, COOH, COO-alkyl, —OZ, —COOZ, a polyethylene glycol group, an alkyl sulfonate group, an alkyl-COOH group, a substituted or unsubstituted benzyl group, a sugar derivative, —C≡C—(CH₂)ₚCO₂R₁₀, where R₁₀ is H or alkyl, and O(CH₂)ᵣCOR₁₁, where R₁₁ is OH, O-alkyl or N-succinimide, and p and r are each independently an integer from 1 to 10; 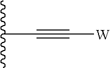
wherein W is an aryl, alkyl or heteroaryl group, each of which may be optionally substituted by one or more substituents selected from OH, CN, CF₃, alkyl, alkoxy, halogen, an isothiocyanate group, a haloacetamide, maleimide, NH₂, NO₂, CONH₂, haloalkyl, COOH, COO-alkyl, —OZ′, —COOZ′, a polyethylene glycol group, an alkyl sulfonate group, an alkyl-COOH group, a substituted or unsubstituted benzyl group, a sugar derivative, —C≡C—(CH₂)_{p′}CO₂R₁₂, where R₁₂ is H or alkyl, and O(CH₂)_{r′}COR₁₃, where R₁₃ is OH, O-alkyl or N-succinimide, and p′ and r′ are each independently an integer from 1 to 10;
where Z and Z′ are each independently silicon-containing protecting groups;
and wherein when a, b, c and d have the members necessary to complete a porphyrin nucleus in which one or two of the nitrogens are replaced by S, O, Se or Te,
(a) R₁, R₂ and R₃ are identical, and R₄≠R₁, R₂, R₃; or
(b) R₁=R₃; R₂=R₄, where R₁, R₃≠R₂, R₄; or
(a) R₂=R₃; R₁≠R₄; andR₁, R₄≠R₂, R₃.

2. A compound of formula Ia 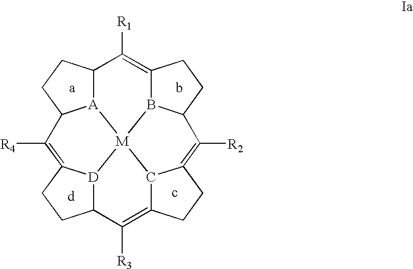
wherein
one or two of A, B, C and D are each independently selected from S, O, Se and Te, and the remainder are N;
a, b, c and d are each independently substituted or unsubstituted 5-membered heterocyclic groups having the members necessary to complete a chlorine bacteriochlorin or isobacteriochlorin nucleus in which one or two of the nitrogens are replaced by S, O, Se or Te;
M is H or ametal;
R₁, R₂, R₃ and R₄ are each independently selected from:
H;
alkyl;
cycloalkyl;
halogen;
aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, —OZ, —COOZ, a polyethylene glycol group, an alkyl sulfonate group, an alkyl-COOH group, a substituted or unsubstituted benzyl group, a sugar derivative, —C≡C—(CH₂)ₚCO₂R₁₀, where R₁₀ is H or alkyl, and O(CH₂)ᵣCOR₁₁ , where R₁₁ is OH, O-alkyl or N-succinimide, and p and r are each independently an integer from I to 10; 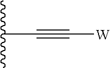
wherein W is an aryl, alkyl or heteroaryl group, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ′, COOZ′, a polyethylene glycol group, an alkyl sulfonate group, an alkyl-COOH group, a substituted or unsubstituted benzyl group, a sugar derivative, —C≡C—(CH₂)_{p′}CO₂R₁₂, where R₁₂ is H or alkyl, and O(CH₂)_{r′}COR₁₃, where R₁₃ is OH, O-alkyl or N-succinimide, and p′ and r′ are each independently an integer from 1 to 10;
where Z and Z′ are each independently silicon-containing protecting groups.

3. A compound according to claim 1 wherein one of A, B, C and D is S and the remainder are all N.

4. A compound according to claim 1 wherein R₁, R₂, R₃ and R₄ are each independently selected from:
H;
halogen;
phenyl or pyridyl, each of which are optionally substituted by one or more substituents selected from OH, CN, CF₃, alkyl, alkoxy, haloalkyl, halogen, NH₂, NO₂, CONH₂, haloalkyl, COOH, COO-alkyl, OZ, COOZ, a polyethylene glycol group, —C≡C—(CH₂)ₚCO₂R₁₀, where R₁₀ is H or alkyl, and O(CH₂)ᵣCOR₁₁, where R₁₁ is OH, O-alkyl or N-succinimide, and p and r are each independently an integer from 1 to 10; 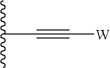
wherein W is a phenyl or pyridyl group, each of which may be optionally substituted by one or more substituents selected from OH, CN, CF₃, alkyl, alkoxy, haloalkyl, halogen, NH₂, NO₂, CONH₂, haloalkyl, COOH, COO-alkyl, OZ′, COOZ′, a polyethylene glycol group, —C≡C—(CH₂)_{p′}CO₂R₁₂, where R₁₂ is H or alkyl, and O(CH₂)_{r′}COR₁₃, where R₁₃ is OH, O-alkyl or N-succinimide, and p′ and r′ are each independently an integer from 1 to 10.

5. A compound according to any preceding claim 1 wherein R₁, R₂, R₃ and R₄ are each independently selected from:
H;
halogen;
phenyl or pyridyl, each of which are optionally substituted by one or more substituents selected from alkoxy, halogen, OH, O(CH₂)ᵣCOR₁₁ and —C≡C—(CH₂)ₚCO₂R₁₀; 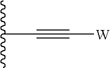
wherein W is phenyl or pyridyl, each of which may be optionally substituted by one or more substituents selected from OH, OZ′ and a polyethylene glycol group.

6. A compound according to claim 1 which is of formula II

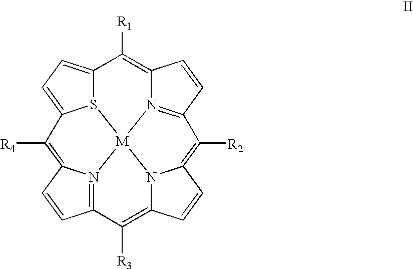

7. A compound according to claim 6 wherein
R₁ and R₄ are different and are selected from aryl and heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOZ; and
R₂ and R₃ are the same and are both H, halogen or

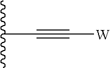

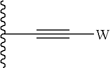

8. A compound according to claim 7 wherein
R₁ is aryl optionally substituted by an alkoxy group;
R₂ and R₃ are both H, halogen or 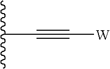
where W is a pyridyl;
R₄ is phenyl.

9. A compound according to claim 8 which is selected from the following:

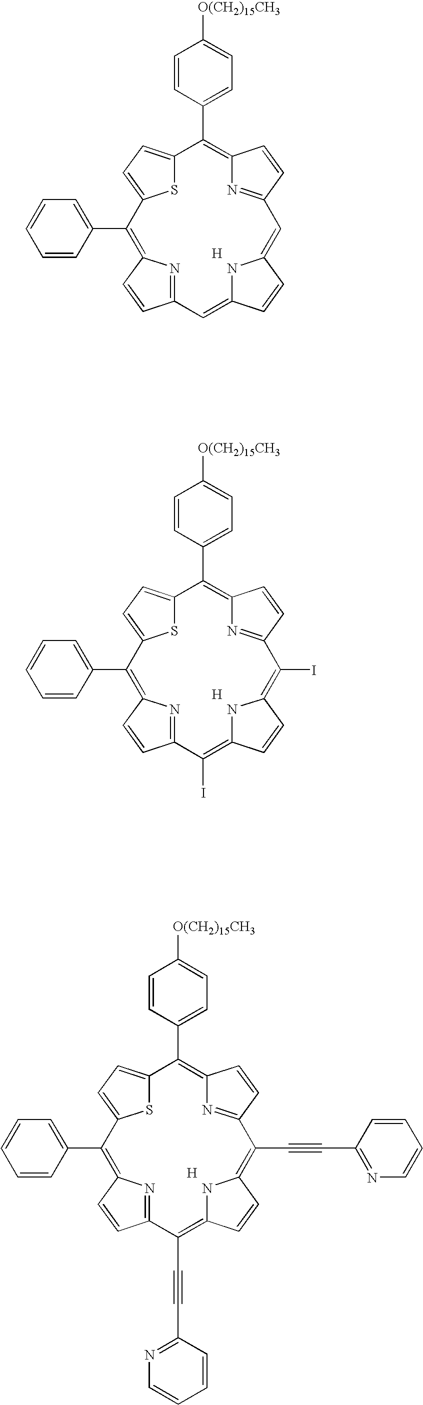

10. A compound according to claim 6 wherein
R₁ are R₃ are the same and are both H, halogen or 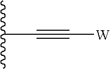
R₂ and R₄ are the same and are both aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOZ.

11. A compound according to claim 10 wherein
R₁ and R₃ are both H, halogen or 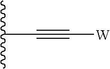
where W is pyridyl;
R₂ and R₄ are both phenyl.

12. A compound according to claim 11 which is selected from the following:

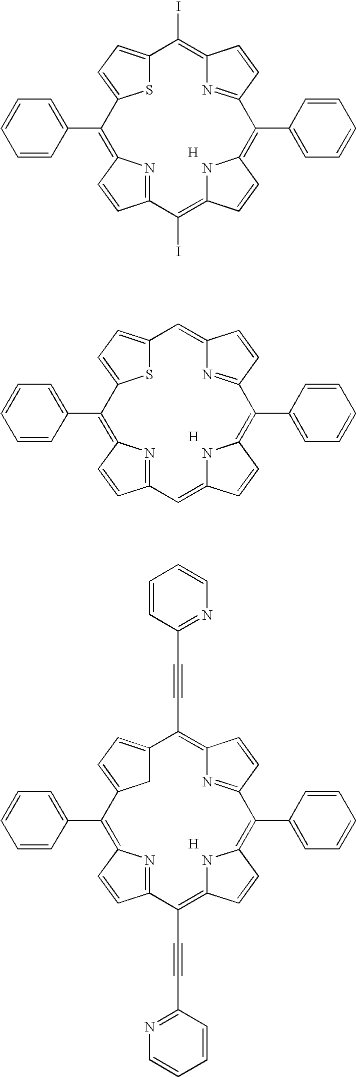

13. A compound according to claim 6 wherein
R₁, R₂ and R₃ are the same and are all H, halogen or 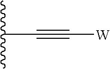
R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ, COOZ, —C≡C—(CH₂)ₚCO₂R₁₀, where R₁₀ is H or alkyl, and O(CH₂)ᵣCOR₁₁, where R₁₁ is OH, O-alkyl or —N-succinimide, and p and r are each independently an integer from 1 to 10.

14. A compound according to claim 13 wherein
R₁, R₂ and R₃ are all H, halogen or 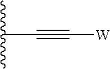
where W is a pyridyl or phenyl group, each of which may be optionally substituted by one or more substituents selected from OH, OZ′, and a polyethylene glycol group; and
R₄ is a phenyl group substituted by one or more halogen, alkoxy, O(CH₂)ₚCOR₁₁ or —C≡C—(CH₂)ₚCO₂R₁₀ groups.

15. A compound according to claim 14 wherein said compound is selected from the following:

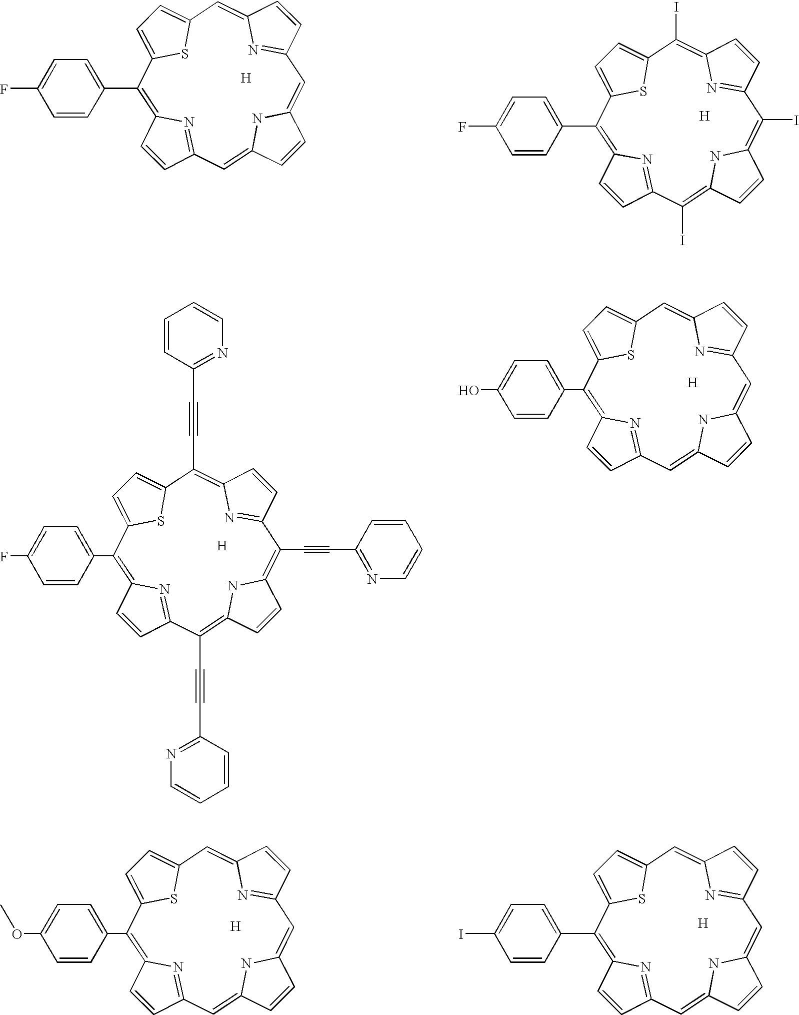

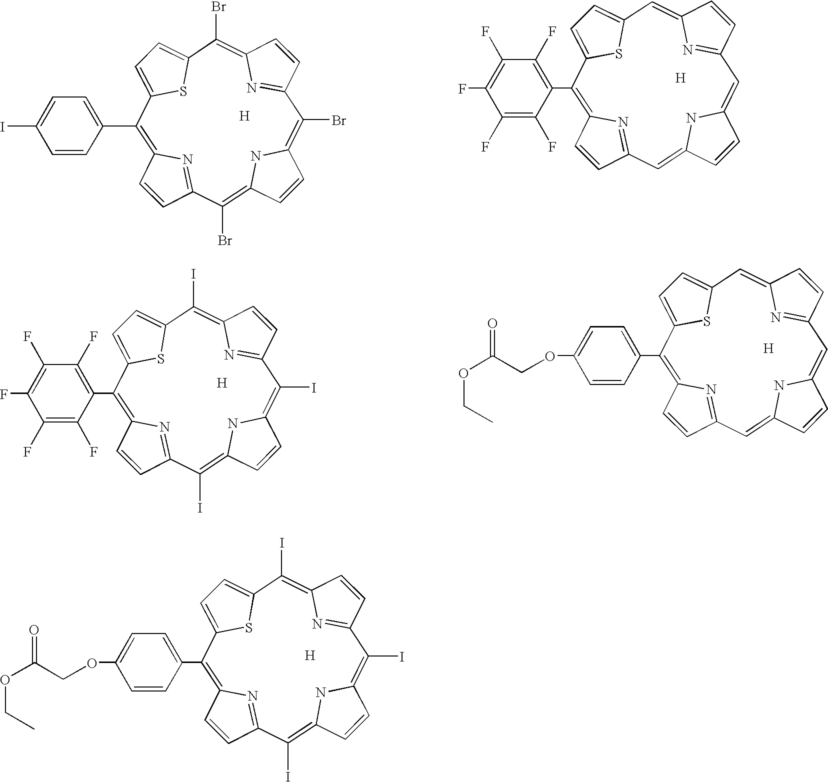

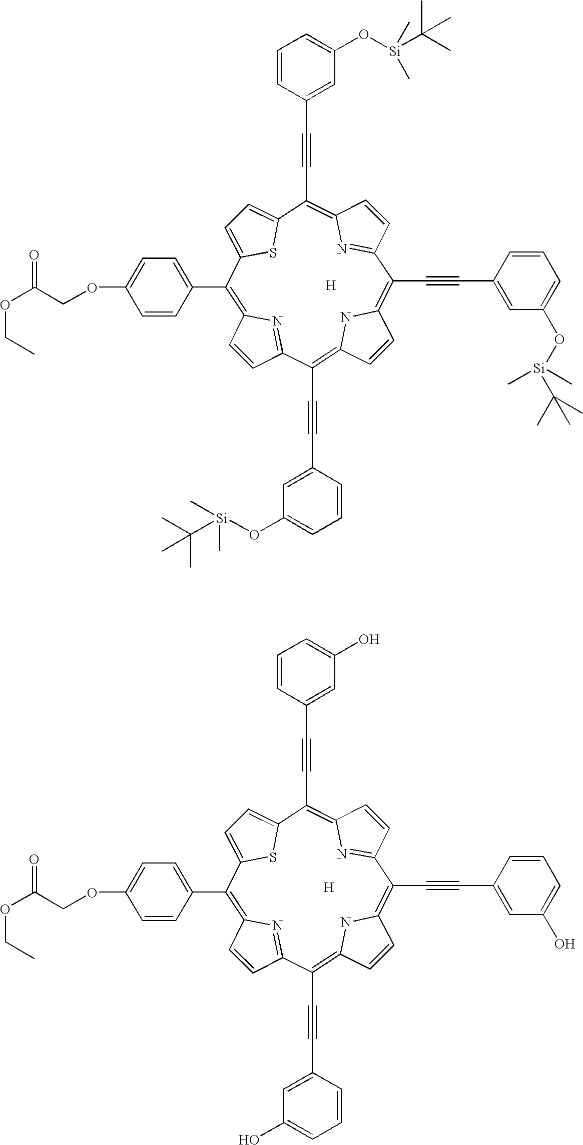

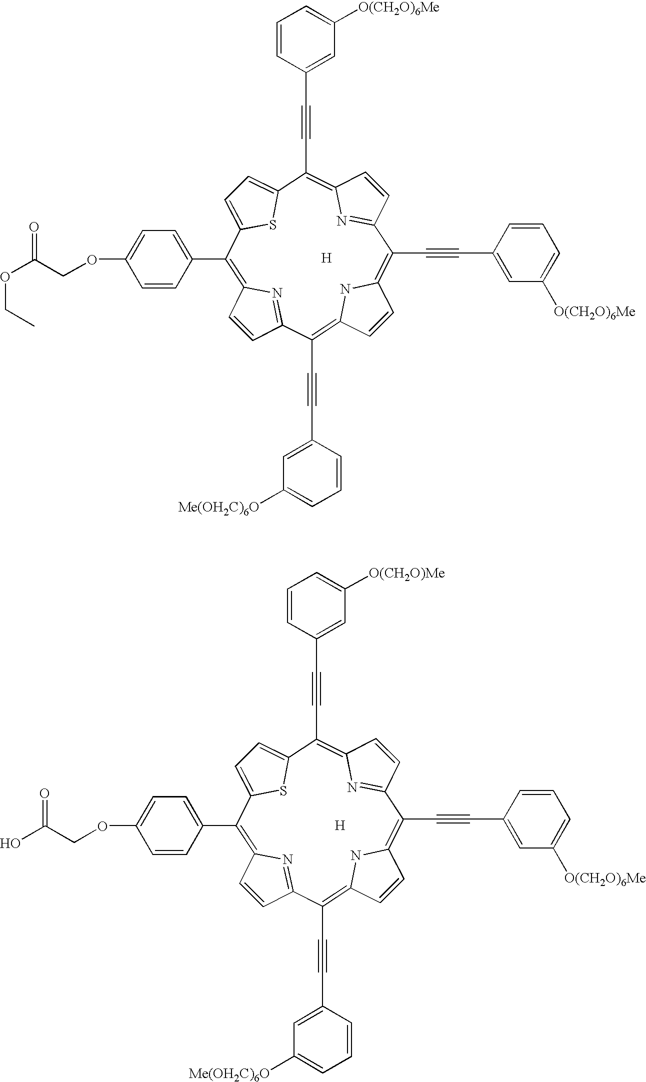

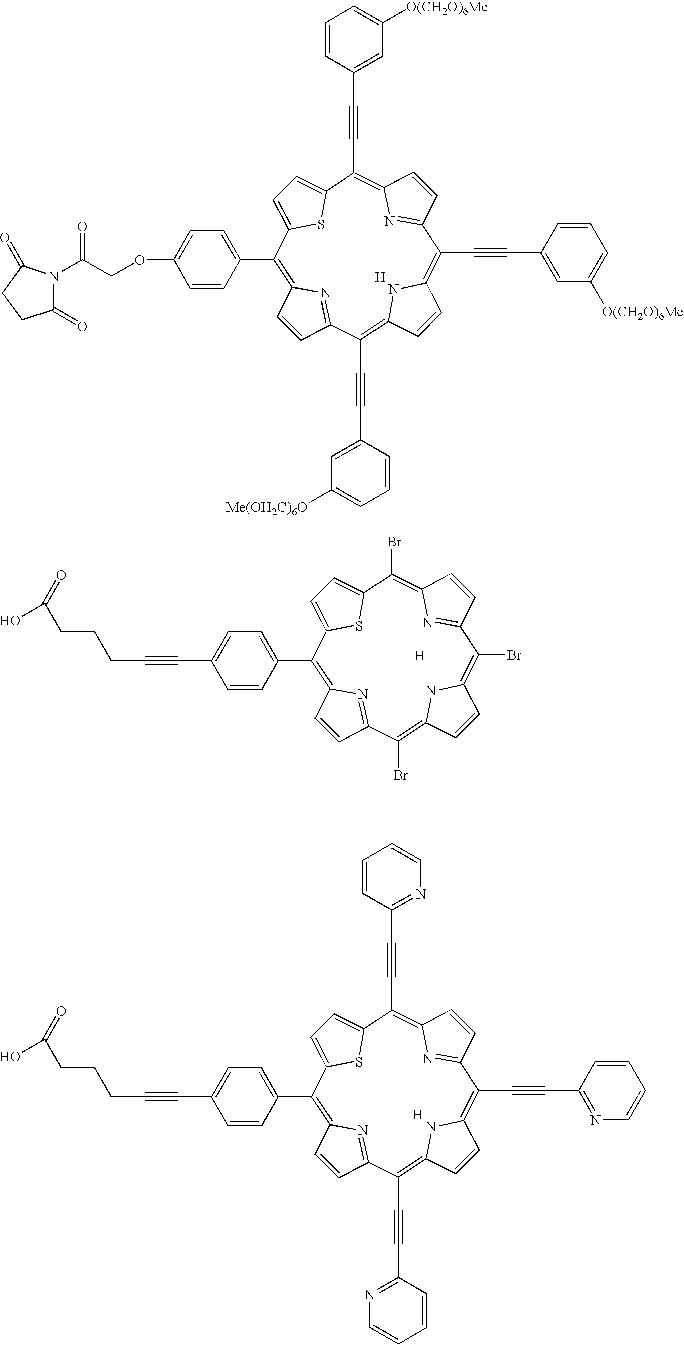

16. A compound according to claim 1 which is of formula III or IV 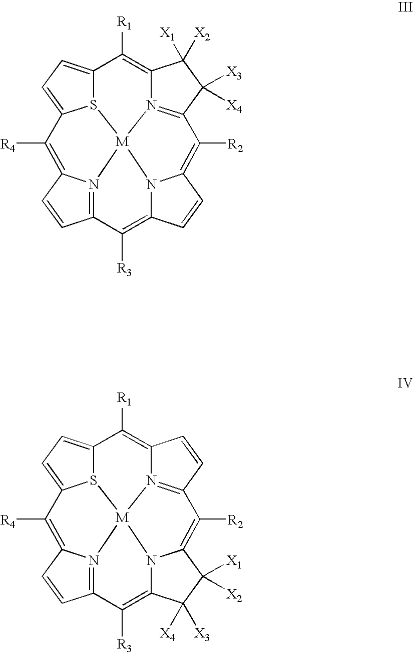
wherein X₁-X₄ are each independently selected from H, OH, alkyl, alkoxy; or C═O, where X₂ and X₄ respectively are absent, and R₁-R₄ and M are as defined in claim 1.

17. A compound according to claim 16 wherein X₁ and X₃ are OH, and X₂ and X₄ are H.

18. A compound according to claim 1 which is of formula V or VI 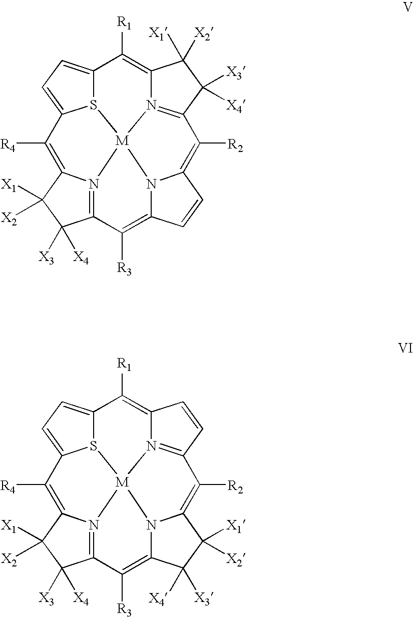
wherein R₁-R₄ and M are as defined in claim 1, and X₁-X₄ and X_{1′}-X_{4′} are each independently selected from H, OH, alkyl, alkoxy; or C═O, where X₂, X₄, X_{2′} and X_{4′} respectively are absent.

19. A compound according to claim 18 wherein X₁, X₃, X₁′ and X₃′ are OH, and X₂, X₄, X₂′ and X₄′ are all H.

20. A compound according to claim 16 wherein
R₁, R₂ and R₃ are the same and are all H, halogen or 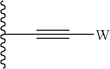
R₄ is aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOz.

21. A compound according to claim 20 wherein
R₁, R₂ and R₃ are all H, halogen or 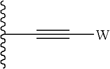
where W is pyridyl; and
R₄ is a halogen substituted aryl group.

22. A compound according to claim 21 which is selected from:

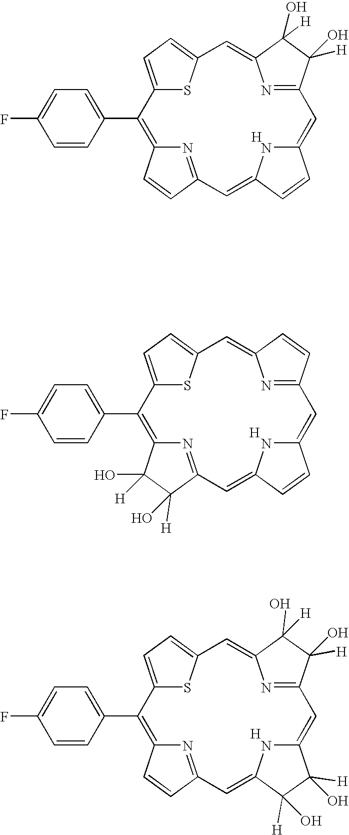

23. A compound according to claim 16 or claim 18 wherein
R₂ and R₃ are the same and are both H, halogen or 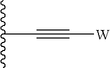
R₁ and R₄ are different and are aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOZ.

24. A compound according to claim 23 wherein
R₂ and R₃ are both H, halogen or 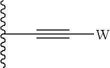
where W is pyridyl;
R₄ is phenyl; and
R₁ is alkoxy substituted phenyl.

25. A compound according to claim 24 which is selected from:

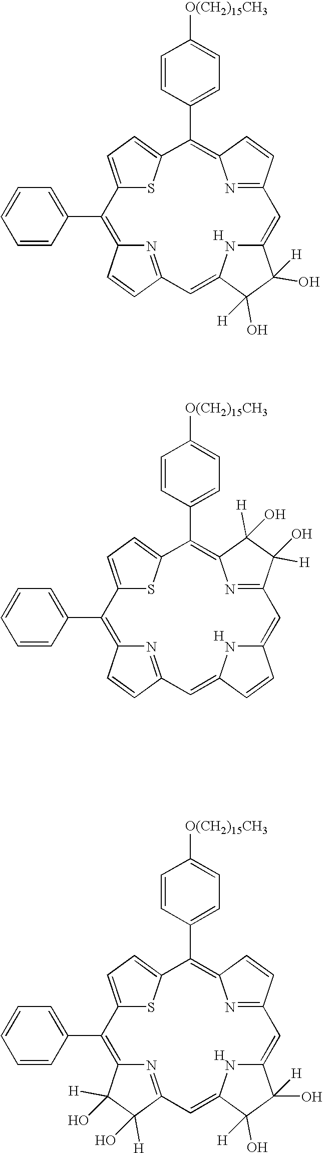

26. A compound according to claim 16 wherein
R₁ and R₃ are the same and are both aryl or heteroaryl, each of which may be optionally substituted by one or more substituents selected from OH, CN, CF₃, alkyl, alkoxy, haloalkyl, halogen, an isothiocyanate group, a haloacetamide, maleimide, NH₂, NO₂, CONH₂, haloalkyl, COOH, COO-alkyl, OZ and COOZ; and
R₂ and R₄ are the same and are both H, halogen or

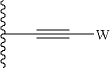

27. A compound according to claim 26 wherein
R₁ and R₃ are both phenyl; and
R₂ and R₄ are both H.

28. A compound according to claim 27 which is selected from the following:

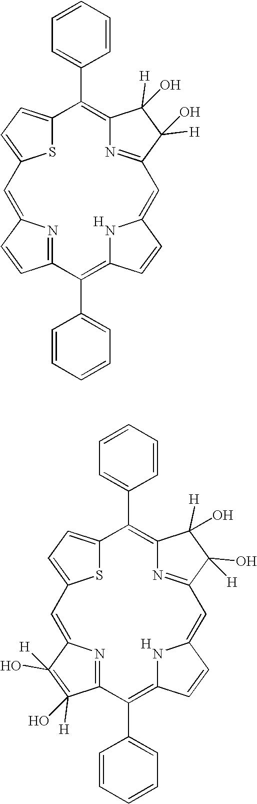

29. A compound according to claim 1 wherein is M is selected from H. Ni, Pb, V, Pd, Co, Nb, Al, Sn, Zn, Cu, Mg, Ca, In, Ga, Fe, Eu, Lu, Pt, Ru, Mn and Ge.

30. A compound according to claim 1 wherein M is H or Zn.

31. A pharmaceutical composition comprising a compound according to claim 1 admixed with a pharmaceutically acceptable diluent, excipient or carrier.

32. A conjugate molecule comprising a compound as defined in claim 1 and a targeting moiety selected from a recombinant antibody, a Fab fragment, a F(ab′)₂ fragment, a single chain Fv, a diabody, a disulfide linked Fv, a single antibody domain and a CDR.

33. A conjugate molecule which comprises a polypeptide carrier comprising at least one alpha helix having synthetically attached thereto a plurality of compounds as defined in claim 1.

34-38. (canceled)

39. A method of treating a proliferative disorder, said method comprising administering to a subject a therapeutic amount of a compound according to claim 1, or a conjugate according to claim 32.

40. A process for preparing a compound as defined in claim 1, said process comprising reacting a compound of formula VII with a dipyrrole to form a compound of formula IX 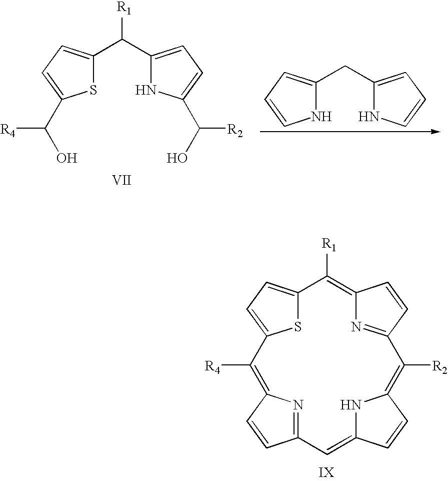
where R₁, R₂ and R₄ are as defined in claim 1.

41. A process according to claim 40 wherein said compound of formula VII is prepared via intermediates X, XI and XII

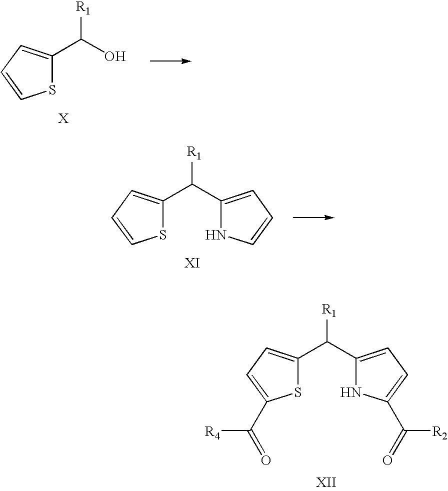

42. A process according to claim 40 for preparing a compound according to claim 17 which further comprises oxidising said compound of formula IX with osmium tetroxide.
